(19) United States
(12) Patent Application Publication
(10) Pub. No.: US 2023/0398241 A1
(43) Pub. Date: Dec. 14, 2023

(54) RADIOLABELED ETHER DENDRIMER CONJUGATES FOR PET IMAGING AND RADIOTHERAPY

(71) Applicant: Ashvattha Therapeutics, Inc., Redwood City, CA (US)

(72) Inventors: Jeffrey L. Cleland, Redwood City, CA (US); Rishi Sharma, Redwood City, CA (US); Minghao Sun, Redwood City, CA (US); Santiago Appiani La Rosa, Redwood City, CA (US)

(73) Assignee: Ashvattha Therapeutics, Inc., Redwood City, CA (US)

(21) Appl. No.: 18/034,551

(22) PCT Filed: Oct. 29, 2021
(86) PCT No.: PCT/US2021/057430

§ 371 Date: Apr. 28, 2023

Related U.S. Application Data
(60) Provisional application No. 63/253,308, filed on Oct. 7, 2021.
(60) Provisional application No. 63/187,851, filed on May 12, 2021.
(60) Provisional application No. 63/108,230, filed on Oct. 30, 2020.

(51) Int. Cl. A61K 51/06; A61K 45/06

(52) U.S. Cl. CPC A61K 51/065; A61K 45/06; A61K 2121/00

(57) ABSTRACT

Compositions and methods for detecting, monitoring and imaging inflammatory sites or tumors in a subject have been developed. Compositions of hydroxyl-terminated dendrimers conjugated to radionuclide(s) via ether linkages are provided for both imaging and radiotherapy (tumors). Methods for the non-invasive and specific positron emission tomography (PET) imaging or magnetic resonance imaging (MRI) of dendrimer conjugated to one or more imaging agents in a subject in vivo are also provided. The methods selectively deliver dendrimer conjugated to radionuclides or MRI contrast agents to reactive microglia or reactive immune cells in tumors in the recipient. In some embodiments, the dendrimers conjugated to imaging agents also deliver targeted radiotherapy agents to a tumor, and/or deliver additional diagnostic, therapeutic or prophylactic agents to reactive microglia in the recipient. Methods of making dendrimers conjugated to imaging agents are also described.

### CROSS-REFERENCE TO RELATED APPLICATIONS

This application claims priority under 35 U.S.C. § 119(e) to U.S. Provisional Patent Application No. 63/253,308, filed Oct. 7, 2021, U.S. Provisional Patent Application No. 63/187,851, filed May 12, 2021, and U.S. Provisional Patent Application No. 63/108,230, filed Oct. 30, 2020, each of which is hereby incorporated by reference in its entirety.

### BACKGROUND

Microglia are the key resident immune cells in the brain that maintain the brain homeostasis by preventing the entry of the pathogens, constantly removing the cell debris from brain parenchyma and repairing neuronal injuries. Microglia/macrophages play a key role after central nervous system (CNS) injury and can have both protective and deleterious effects based on the timing and type of insult. Microglial activation is a major pathological event in early brain injury which leads to neuronal injury and further disease progression (Li, et al., Nature Reviews Immunology, 2017, 18, 225; Hernadez-Ontiveros, et al., Frontiers in Neurology 2013, 4 (30); Ramlackhansingh, et al., Annals of neurology 2011, 70 (3), 374-383).

The development of smart, specific and non-invasive preclinical and clinical imaging probes to track activated microglia is required. In particular, there is a need for tools and systems to assess the efficacies of new neuroinflammatory therapies in vivo. However, the development of nanoprobes for imaging of neuroinflammation must overcome challenges relating to the blood-brain barrier (BBB), brain tissue penetration, and specificity for targeting of reactive microglia.

Positron emission tomography (PET) is widely applied in clinical oncology for cancer diagnosis, neurology for brain function evaluation, cardiology for heart function evaluation, and in infectious diseases. Translocator protein 18 kDa (TSPO) is the most investigated biomarker for PET imaging of the brain, and several TSPO ligand-based PET tracers are currently in use. However, the lack of cellular specificity of TSPO poses challenges with the quantitation necessitating the need for activated microglia-specific highly sensitive PET probes (Vaquero, et al, Annu Rev Biomed Eng 2015, 17, 385-414; Narayanaswami, et al., Mol Imaging 2018, 17, 1536012118792317-1536012118792317; Janssen, et al., Academic Press: 2019; Vol. 165, pp 371-399; Werry, et al., Int J Mol Sci 2019, 20 (13), 3161; Papadopoulos, Experimental Neurology 2009, 219 (1), 53-57).

Other imaging technique include computed tomography (CT) and Magnetic Resonance Imaging (MRI). Magnetic Resonance Imaging (MRI) uses radio-waves in the presence of a strong magnetic field that surrounds the opening of the MRI machine where the patient lies to get tissues to emit radio waves of their own. Different tissues (including tumors) emit a more or less intense signal based on their chemical makeup, so a picture of the body organs can be displayed on a computer screen. Much like CT scans, MRI can produce three-dimensional images of sections of the body, but MRI is sometimes more sensitive than CT scans for distinguishing soft tissues.

Accordingly, in some aspects, the disclosure provides compositions and methods for non-invasive detection of sites of inflammation or neuroinflammation in a subject, and methods of making and use thereof. In some aspects, the disclosure provides compositions for in vivo molecular imaging of cancer cells, such as metastatic cancer cells, and methods of making and use thereof. In some aspects, the disclosure provides compositions and methods for selectively targeting radiotherapy to cancer cells.

### SUMMARY

Aspects of the disclosure relate to the discovery that dendrimers (e.g., hydroxyl PAMAM dendrimers) conjugated or complexed with radionuclides, such as ¹⁸F (Fluorine-18), ⁸⁹Zr (Zirconium-89), ⁹⁰Y (Yttrium-90), and ¹⁷⁷Lu (Luthenium-177), can cross the BBB in the presence of reactive microglia, and selectively target these microglia after systemic administration. In some embodiments, these conjugates are stable Positron emission tomography (PET) imaging probes for the non-invasive and specific imaging of reactive microglia in vivo and stable radiotherapy agents for the treatment of tumors. In some aspects, the disclosure provides compositions and methods for detecting one or more inflammatory sites in a subject in need thereof. In some aspects, the disclosure provides compositions and methods for treating cancer in a subject in need thereof.

In some aspects, the disclosure provides compositions of hydroxyl-terminated dendrimers conjugated to one or more imaging agents via ether linkages. In some embodiments, hydroxyl-terminated dendrimers are conjugated to imaging agents, where imaging agents can include radionuclide or MIRI contrast agents. In some embodiments, the dendrimer conjugates selectively accumulate within reactive microglia at sites of inflammation and in reactive immune cells in tumor as stable imaging probes in vivo or radiotherapeutics. In some embodiments, the dendrimer conjugates also treat the site of inflammation or cancer, for example, by selectively delivering radiotherapeutic agents to the site of inflammation or cancer. In other embodiments, the dendrimer conjugates include one or more additional active agents. Therefore, in some embodiments, the dendrimer conjugates deliver one or more additional active agents in vivo to sites of inflammation, neuroinflammation, or tumor with enhanced stability. In some embodiments, the radionuclides and/or the MRI contrast agents are attached to the dendrimer via an ether linkage.

In some aspects, the disclosure provides a composition comprising a compound that comprises a dendrimer conjugated to a radionuclide or an MRI contrast agent through an ester, ether, or amide linkage. In some embodiments, the dendrimer comprises a high density of surface hydroxyl groups. In some embodiments, the dendrimer is conjugated to the radionuclide or the MRI contrast agent through an ether or amide linkage. In some embodiments, the dendrimer is conjugated to the radionuclide or the MRI contrast agent through an ether linkage.

In some embodiments, the radionuclide or the MRI contrast agent is conjugated to the ester, ether, or amide linkage through a spacer. In some embodiments, the spacer comprises alkyl groups, heteroalkyl groups, and/or alkylaryl groups. In some embodiments, the spacer comprises a peptide. In some embodiments, the spacer comprises polyethylene glycol.

In some embodiments, conjugation of the radionuclide or the MRI contrast agent occurs on less than 50% of total available surface functional groups of the dendrimer prior to the conjugation. In some embodiments, conjugation of the radionuclide or the MRI contrast agent occurs on less than 5%, less than 10%, less than 20%, less than 30%, or less than 40% of total available surface functional groups of the dendrimer prior to the conjugation.

In some embodiments, the radionuclide is selected from the group consisting of ¹⁸F, ⁵¹Mn, ⁵²Fe, ⁶⁰Cu, ⁶⁸Ga, ⁷²As, ^{94m}Tc, ¹¹⁰In, ¹⁸F, ¹²⁴I, ¹²⁵I, ¹³¹I, ¹²³I, ⁷⁷Br, ⁷⁶Br, ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y ⁸⁰Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ²²⁵Ac, ¹⁹⁸Au, ¹⁹⁹Au, and, ⁸⁹Zr. In some embodiments, the radionuclide is, ¹⁸F, ⁸⁹Zr, ⁹⁰Y, or ¹⁷⁷Lu. In some embodiments, the MRI contrast agent is selected from the group consisting of Gd, Mn, BaSO₄, iron oxides, and iron platinum. In some embodiments, the MRI contrast agent is Gd.

In some embodiments, the dendrimer is selected from the group consisting of polyamidoamine (PAMAM) dendrimers, polypropylamine (POPAM) dendrimers, polyethylenimine dendrimers, polylysine dendrimers, polyester dendrimers, iptycene dendrimers, aliphatic poly(ether) dendrimers, and aromatic polyether dendrimers.

In some embodiments, the zeta potential of the compound is between −25 mV and 25 mV. In some embodiments, the zeta potential of the compound is between −20 mV and 20 mV, between −10 mV and 10 mV, between −10 mV and 5 mV, between −5 mV and 5 mV, or between −2 mV and 2 mV. In some embodiments, the surface charge of the compound is neutral or near-neutral.

In some aspects, the disclosure provides methods for detecting one or more inflammatory sites in a subject in need thereof. In some aspects, the disclosure provides methods for imaging one or more inflammatory sites in a subject in need thereof. In some embodiments, the methods comprise administering to the subject an effective amount of a composition described herein. For example, in some embodiments, the composition comprises a compound comprising a dendrimer conjugated to a radionuclide or an MRI contrast agent through an ester, ether, or amide linkage, where the dendrimer comprises a high density of surface hydroxyl groups.

In some embodiments, the methods selectively deliver one or more radionuclides or magnetic resonance imaging (MRI) contrast agents to the target sites of the recipient. In some embodiments, the methods include administering to a subject a formulation including hydroxyl-terminated dendrimers conjugated to one or more radionuclides or MRI contrast agents via ether linkages. Exemplary radionuclides that can be delivered include ¹⁸F, ⁵Mn, ⁵²Fe, ⁶⁰Cu, ⁶⁸G, ⁷²As, ^{94m}Tc, or ¹¹⁰In, ¹⁸F, ¹²⁴I, ¹²⁵I, ¹³¹I, ¹²³I ⁷⁷Br, ⁷⁶Br, ^{99m}Tc ⁵¹Cr, ⁶⁷G, ⁶⁸G, ⁴⁷Sc, ⁵¹Cr, ¹⁶⁷Tm, ¹⁴¹Ce, ¹¹¹In, ¹⁶⁸Yb ¹⁷⁵Yb ¹⁴⁰La, ⁹⁰Y, ⁸⁰Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ²²⁵Ac, ¹⁹⁸Au and ¹⁹⁹Au, or ⁸⁹Zr. Exemplary MRI contrast agents that can be delivered include Gd, Mn, BaSO₄, iron oxides, and iron platinum.

In some embodiments, the methods deliver one or more radionuclides or MRI contrast agents to the subject in an amount effective to achieve a physiological response in the subject. The dendrimers are optionally complexed to, covalently conjugated to, or having intra-molecularly dispersed or encapsulated therein one or more additional active agents, such as therapeutic or prophylactic agents. In some embodiments, the formulation is administered systemically to a subject, in an amount effective to detect, diagnose or monitor one or more inflammatory sites or cancer in the recipient, and/or to treat, alleviate or prevent one or more symptoms of the inflammation or cancer in the subject. Exemplary inflammatory sites or cancers that can be detected, diagnosed or monitored and/or treated include sites of autoimmune diseases or disorder, sites of neuroinflammation in the brain, and solid tumors, including brain tumors. Additional exemplary inflammatory sites include those associated with one or more inflammatory diseases, or associated with sepsis or septic shock, or caused by any mechanism of macrophage activation including macrophage activation syndrome. In some embodiments, the one or more inflammatory sites in the subject include one or more sites of neuroinflammation in the central nervous system. In some embodiments, the one or more sites of neuroinflammation in the central nervous system in the subject are associated with Alzheimer's disease. In some embodiments, the one or more inflammatory sites in the subject are associated with amyotrophic lateral sclerosis (ALS). In some embodiments, the dendrimers are generation 4, generation 5, or generation 6 poly(amidoamine) (PAMAM) hydroxyl-terminated dendrimers.

In some aspects, the disclosure provides compositions and methods for detecting cancer cells in a subject in vivo. In some embodiments, the method involves administering to the subject one or more hydroxyl-terminated dendrimers conjugated or complexed with one or more radionuclides and/or one or more MRI contrast agents, that selectively target a tumor region. In some embodiments, the subject can then be imaged with a molecular imaging device to detect the hydroxyl-terminated dendrimers conjugated or complexed with one or more radionuclides in the subject. In some embodiments, the subject can also be imaged with an MRI scanner to detect the hydroxyl-terminated dendrimers conjugated or complexed with one or more MRI contrast agents in the subject. Thus, in some embodiments, detection of the labeled hydroxyl-terminated dendrimers in an organ or tissue of a subject can be an indication of cancer cells in the organ.

In some embodiments, the cancer cells can be primary tumors or metastasized cancer cells. Therefore, in some embodiments, the methods involve administering dendrimers conjugated or complexed with one or more radionuclides to a subject diagnosed with a primary tumor to identify metastasized cancer cells. In other embodiments, the method involves administering dendrimers conjugated or complexed with one or more radionuclides to a subject at risk of cancer to detect primary or occult tumors. Non-limiting examples of cancer cells that can be detected by the disclosed methods include renal cell carcinoma.

In some aspects, the disclosure provides a method for treating a neurodegenerative disorder. In some embodiments, the method comprises administering to a subject in need thereof an effective amount of a composition described herein. In some embodiments, the composition comprises a compound comprising a dendrimer conjugated to a radionuclide or an MRI contrast agent through an ester, ether, or amide linkage, where the dendrimer comprises a high density of surface hydroxyl groups. In some embodiments, the neurodegenerative disorder is Alzheimer's disease. In some embodiments, the neurodegenerative disorder is ALS.

Exemplary devices for use in molecular imaging include, for example, devices for positron emission tomography (PET) scanning; devices for computed tomography (CT) and nuclear medicine imaging; devices for magnetic resonance imaging (MRI). In some embodiments, the molecular imaging devices are a gamma camera suitable for positron emission tomography (PET) scanning and an MRI scanner.

In some embodiments, the formulation can be formulated for intravenous administration to the subject or for enteral administration. In some embodiments, the formulation is administered prior to, in conjunction with, subsequent to, or in alternation with treatment with one or more additional procedures or therapies. Exemplary additional procedures include administering one or more therapeutic, prophylactic and/or diagnostic agents to prevent or treat one or more symptoms of associated diseases or conditions of inflammation, neuroinflammation, and cancers.

In some aspects, the disclosure provides pharmaceutical formulations of hydroxyl dendrimers complexed to, covalently conjugated to, or encapsulated therein one or more radionuclides. In some aspects, the disclosure provides kits including the hydroxyl dendrimers complexed to, covalently conjugated to, or encapsulated therein one or more radionuclides or one or more MRI contrast agents.

In some aspects, the disclosure provides methods of making hydroxyl dendrimers complexed to, covalently conjugated to, or encapsulated therein one or more radionuclides.

### BRIEF DESCRIPTION OF THE DRAWINGS

FIG. 1 is a scheme showing synthetic route for ¹⁸F labeled hydroxyl dendrimer. Reagents and conditions: (i): DMSO, 2% NaOH solution, rt, ON; (ii): CuBr(I), N,N,N′,N″,N″-Pentamethyldiethylenetriamine, 2 h, 60° C.; (iii): KF, K₂CO₃, Cryptand 222, 105° C., 20 min., DMSO.

FIG. 2 is a bar graph showing in vitro plasma stability of fluorinated dendrimer showing highly stable conjugate with negligible defluorination.

FIG. 3 is a scheme showing synthetic route for ⁸⁹Zr labeled hydroxyl dendrimer. Reagents and conditions: (i): Allyl Bromide, CS₂CO₃, TBAI, DMF, rt, ON; (ii): Cysteamine, 2,2-Dimethoxy-2-phenylacetophenone, DMF, 365 nm, 8 h, rt; (iii): SCN-Bn-Deferoxamine, DIPEA, DMSO, rt, ON; (iv): ⁸⁹Zr⁴⁺, HEPES buffer, pH 7.5, rt, 60 min.

FIG. 4 is a bar graph showing in vitro plasma stability of Zr-labeled dendrimer showing highly stable conjugate with negligible release.

FIG. 5 is a scheme showing synthetic route for ⁹⁰Y labeled hydroxyl dendrimer. Reagents and conditions: (i): DMF, CuSO₄·5H₂O, sodium ascorbate rt, ON; (ii): DMSO, DIPEA, RT, pH 7.5, ON; (iii): Yttrium chloride, ammonium acetate buffer (0.5 M, pH ˜6.5-7.5), 100° C., 30-60 min.

FIG. 6 is a scheme showing synthetic route for ⁹⁰Y labeled hydroxyl dendrimer-antitumor combination. Reagents and conditions: (i): DMF, CuSO₄·5H₂O, sodium ascorbate rt, ON; (ii): DMSO, DIPEA, RT, pH 7.5, ON; (iii): Yttrium chloride, ammonium acetate buffer (0.5 M, pH ˜6.5-7.5), 100° C., 30-60 min.

FIGS. 7A-7E show results from imaging neuroinflammation and neurodegenerative disease using ¹⁸F labeled dendrimer. FIG. 7A depicts a summary of the extent of hydroxyl dendrimer localization in reactive microglia, as a function of blood brain barrier (BBB) impairment in multiple CNS disorder models. FIG. 7B shows images depicting the histology of brain sections containing beta amyloid plaque from animals sacrificed at 48 hours post-dose and measured by confocal fluorescence microscopy, showing selective uptake of Cy5 labelled hydroxyl dendrimer after single IV administration (55 mg/kg) in 7 month old 5×FAD mice. FIG. 7C shows images representative of summed brain PET/CT images 50-60 minutes after tracer administration and 24 hours after injection of saline or LPS (10 mg/kg); mice injected with LPS displayed varying murine sepsis scores indicative of their symptom severity, and PET imaging shows mice with low and high sepsis scores and demonstrate the ability of ¹⁸F-OP-801 to detect increasing levels of inflammation in a manner that correlates with symptom severity. FIG. 7D shows images demonstrating whole body PET/CT images, which revealed markedly higher uptake of ¹⁸F-OP-801 in LPS-injected mice compared to those given saline alone. FIG. 7E is a line graph showing group average time-activity curve (TCA) of whole brain measure as % ID/g over a period of 60 minutes in saline-injected or LPS-injected mice. FIG. 7F is a bar graph showing quantitation of 50-60 minute summed brain PET images, showing significantly elevated ¹⁸F-OP-801 uptake in cortex, medulla, olfactory bulb, and pons of LPS- (n=10) and saline-injected (n=6) female mice *: p<0.05 **: p<0.01. FIGS. 7G and 7H are bar graphs showing 25-35 min summed ¹⁸F-OP-801 PET quantitation in organs including liver and lung (FIG. 7G) and whole brain and hippocampus (FIG. 7H) of LPS-injected (n=6) and saline-injected (n=5) mice. *: p=0.045. FIG. 7I is a bar graph showing Ex vivo ¹⁸F-OP-801 biodistribution 70 min after injection in LPS-injected (n=4, 2 low-MSS scoring mice excluded) and saline-injected (n=5) mice. FIG. 7J shows imaging from autoradiography of 40 m-thick sagittal brain slices from representative saline versus high-MSS LPS mouse, 70 minutes after injection, overlaid on nissl stain of same slice. FIG. 7K is a plot of LPS MSS scores versus % ID/g in whole brain from 50-60 minute PET, with linear regression and 95% confidence intervals shown. FIG. 7L is a bar graph showing plasma stability of the ¹⁸F labeled hydroxyl dendrimer (¹⁸F-OP-801), n=4 female mice per time point. FIGS. 7M and 7N show a comparison of PET imaging with ¹⁸F-GE180 (FIG. 7M) and ¹⁸F-OP-801 (FIG. 7N) in 3.75 month old 5×FAD mice and age-matched wild type controls. FIG. 7O shows a comparison of PET imaging with ¹⁸F-OP-801 in 5 month old 5×FAD mice and age-matched wild type controls.

FIG. 8 shows the synthesis of D6-B483.

FIGS. 9A-9B show selective uptake of ¹¹¹In-D6-B483 in brain and solid tumors.

FIG. 10 shows localization of ¹¹¹In-D6-B483 in large tumors.

FIG. 11 shows localization of ¹¹¹In-D6-B483 in small tumors.

### DETAILED DESCRIPTION OF THE INVENTION

Among other aspects, the disclosure relates to the discovery that hydroxylated dendrimers, such as hydroxyl PAMAM dendrimers, complexed or conjugated with radionuclides, such as ¹⁸F (Fluorine-18) and/or ⁸⁹Zr (Zirconium-89), can cross the impaired BBB in the absence of any targeting ligand, and selectively target activated microglia upon systemic administration as stable Positron emission tomography (PET) imaging probes for the non-invasive and specific imaging of activated microglia. The hydroxylated dendrimers (e.g., hydroxyl PAMAM dendrimers) complexed or conjugated with radionuclides, such as ¹⁸F or ⁸⁹Zr, are not observed in the brain cells of healthy brains. Without wishing to be bound by theory, the mechanism for selective uptake is thought to be related to the ability of hydroxyl dendrimers complexed or conjugated with radionuclides, such as ¹⁸F or ⁸⁹Zr, to diffuse well within the brain, for uptake by increasingly phagocytic activated glia. The hydroxyl dendrimers complexed or conjugated with ¹⁸F or ⁸⁹Zr are nontoxic, even at intravenous doses >500 mg/kg, and are cleared intact through the kidney.

Accordingly, in some aspects, the disclosure provides compositions of dendrimer complexes comprising dendrimers complexed or conjugated with radionuclides, such as ¹⁸F (Fluorine-18) or ⁸⁹Zr to diagnose, detect, and/or image a site of inflammation, e.g., neuroinflammation, via PET imaging in a subject in need thereof. In some embodiments, the compositions of dendrimer complexes with ¹⁸F or ⁸⁹Zr are suitable for delivering one or more active agents, particularly one or more active agents to diagnose, detect, and/or image a site of inflammation, e.g., neuroinflammation, in a subject in need thereof. In some embodiments, the compositions are also suited for diagnosing, detecting, and/or imaging cancer cells in vivo, and/or treating or ameliorating one or more symptoms associated with the cancer.

In some aspects, the disclosure relates to the discovery that hydroxyl-terminated dendrimers complexed or conjugated with radionuclides, such as ¹⁸F or ⁸⁹Zr, and further conjugated to one or more active agents via ether linkages, have enhanced stability in vivo. The inventors have recognized and appreciated that hydroxyl-terminated dendrimers labeled with ¹⁸F or ⁸⁹Zr, and further conjugated to one or more active agents via ether linkages, selectively deliver the active agents in vivo to sites of inflammation, neuroinflammation, and/or tumor.

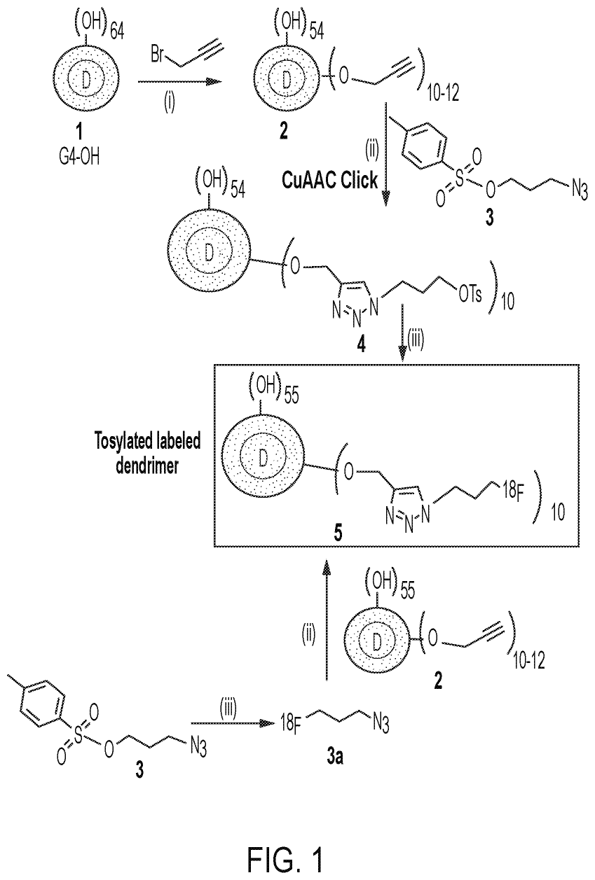

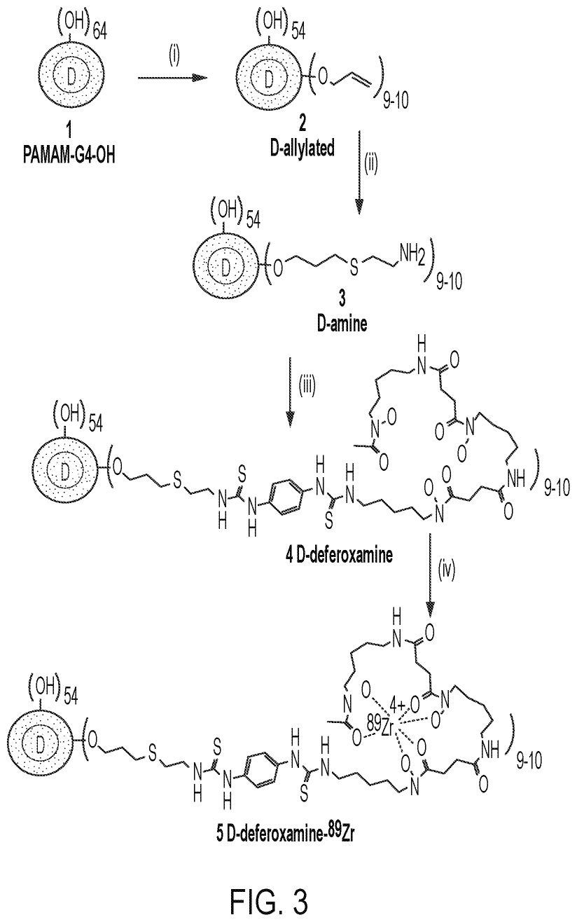

Accordingly, in some aspects, the disclosure provides compositions of hydroxyl-terminated dendrimers complexed or conjugated with radionuclides, such as with ¹⁸F or ⁸⁹Zr, including one or more prophylactic, therapeutic, and/or diagnostic agents complexed and/or conjugated in the dendrimers. In some embodiments, one or more active agent is complexed and/or conjugated in the dendrimer complex at a concentration of about 0.01% to about 30% by weight, e.g., about 1% to about 20% by weight, about 5% to about 20% by weight. In some embodiments, hydroxyl groups of hydroxyl-terminated dendrimers are covalently conjugated to one or more active agents via at least one ether linkage, optionally via one or more linkers/spacers. In some embodiments, surface groups of hydroxyl-terminated dendrimers are modified via etherification reaction prior to conjugation to one or more linkers and the active agent. In some embodiments, where one or more linkers are present between dendrimers and active agents, the covalent bond between the surface groups of dendrimers and the linkers are ether bonds, for example, as shown in FIGS. 1 and 3.

The presence of the additional agents can affect the zeta-potential or the surface charge of the particle. In some embodiments, the zeta potential of the dendrimers is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. In some embodiments, the surface charge is neutral or near neutral. The range above is inclusive of all values from −100 mV to 100 mV.

### Dendrimers

Dendrimers are three-dimensional, hyperbranched, monodispersed, globular and polyvalent macromolecules including a high density of surface end groups (Tomalia, D. A., et al., Biochemical Society Transactions, 35, 61 (2007); and Sharma, A., et al., ACS Macro Letters, 3, 1079 (2014)).

In the past few decades, dendrimers acquired significant attention from the scientific community, especially in the field of targeted drug delivery, due to their precisely well-defined hyperbranched and multivalent architecture. These tree-like globular macromolecules, by virtue of their structure, can be efficiently developed in a highly controlled synthetic manner where surface groups can be easily manipulated to introduce a variety of ligands and other therapeutically relevant bioactive molecules. Due to their unique structural and physical features, dendrimers are useful as nano-carriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Dendrimer surface groups have a significant impact on their biodistribution (Nance, E., et al., Biomaterials, 101, 96 (2016)). Hydroxyl terminated generation 4 PAMAM dendrimers (˜4 nm in size), without any targeting ligand, cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013)).

In some embodiments, the term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

In some embodiments, dendrimers have a diameter between about 1 nm and about 50 nm, between about 1 nm and about 20 nm, between about 1 nm and about 10 nm, or between about 1 nm and about 5 nm. In some embodiments, the diameter is between about 1 nm and about 2 nm. Conjugates are generally in the same size range, in some embodiments, although large proteins such as antibodies may increase the size by 5-15 nm. In some embodiments, an agent is encapsulated with or conjugated to a dendrimer in a ratio of agent to dendrimer of between 1:1 and 4:1 for the larger generation dendrimers. In some embodiments, the dendrimers have a diameter effective to target hepatocytes and to retain in hepatocytes for a prolonged period.

In some embodiments, dendrimers have a molecular weight between about 500 Daltons and about 100,000 Daltons, between about 500 Daltons and about 50,000 Daltons, or between about 1,000 Daltons and about 20,000 Daltons. The molecular weight is a function of the monomer molecular weight.

Suitable dendrimers scaffolds that can be used include poly(amidoamine), also known as PAMAM, or STARBURST™ dendrimers, polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In some embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means a poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In some embodiments, the dendrimers are soluble in the formulation and are generation (“G”) 4, 5 or 6 dendrimers (i.e., G4-G6 dendrimers), and/or G4-G10 dendrimers, G6-G10 dendrimers, or G2-G10 dendrimers. The dendrimers may have hydroxyl groups attached to their functional surface groups. In some embodiments, the dendrimers are generation 4, generation 5, generation 6, generation 7, or generation 8 hydroxyl terminated dendrimers (e.g., poly(amidoamine) dendrimers).

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO 2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, e.g., hepatocytes. In some embodiments, the dendrimer specifically targets a particular tissue region and/or cell type without a targeting moiety.

In some embodiments, the dendrimers have a plurality of hydroxyl (—OH) groups on the periphery of the dendrimers. The surface density of hydroxyl (—OH) groups, in some embodiments, is at least 1 OH group/nm² (number of hydroxyl surface groups/surface area in nm²). For example, in some embodiments, the surface density of hydroxyl groups is more than 2, 3, 4, 5, 6, 7, 8, 9, 10 OH groups/nm²; at least 10, 15, 20, 25, 30, 35, 40, 45, 50, or more than 50 OH groups/nm². In further embodiments, the surface density of hydroxyl (—OH) groups is between about 1 and about 50 OH groups/nm², or 5-20 OH groups/nm² (number of hydroxyl surface groups/surface area in nm²), while having a molecular weight of between about 500 Da and about 10 kDa.

In some embodiments, the dendrimers may have a fraction of the hydroxyl groups exposed on the outer surface, with the others in the interior core of the dendrimers. In some embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10 OH groups/nm³, or more than 10, 15, 20, 25, 30, 35, 40, 45, and 50 OH groups/nm³. In some embodiments, the volumetric density of hydroxyl groups is between about 4 and about 50 OH groups/nm³, between about 5 and about 30 OH groups/nm³, between about 10 and about 20 OH groups/nm³. The diameter of a dendrimer can be generally determined by transmission electron and atomic force microscopies, dynamic light scattering, computations, or a combination thereof for determining the surface area and the volume.

In some embodiments, the dendrimers include an effective number of hydroxyl groups for targeting to activated microglia with a disease, disorder, or injury of the CNS, a site of inflammation, or a tumor region.

Dendrimer Complexes with Imaging Agents

Complexes of dendrimers conjugated with one or more diagnostic or imaging agents are described. The dendrimers can be conjugated with a radionuclide reporter appropriate for scintigraphy, SPECT, or PET imaging and/or with a radionuclide appropriate for radiotherapy; or an MRI contrast agent for MRI imaging. Dendrimer complexes in which the dendrimers are conjugated with both a chelator for a radionuclide or an MRI contrast agent useful for diagnostic imaging and a chelator useful for radiotherapy are specifically contemplated, and in some embodiments, the conjugation is via ether linkages. Therefore, in some embodiments, a singular dendrimer/imaging agent composition can simultaneously treat and/or diagnose a disease or a condition at one or more locations in the body. Also disclosed are radioactively labeled SPECT, or scintigraphic imaging agents that have a suitable amount of radioactivity.

Suitable imaging agents can be selected based on the choice of imaging method. For example, in some embodiments, the imaging agent is near infrared fluorescent dye for optical imaging, a Gadolinium chelate for MRI imaging, a radionuclide for PET or SPECT imaging, or a gold nanoparticle for CT imaging.

Compositions of dendrimers conjugated or complexed with one or more imaging probes for Positron emission tomography (PET) including one or more radionuclides are provided.

PET is a technique that uses a special camera and a computer to detect small amounts of radioactive radiotracers or radiopharmaceuticals in vivo, to evaluate organ and tissue functions. PET can detect the early onset of disease before other tests can.

PET involves the detection of gamma rays in the form of annihilation photons from short-lived positron emitting radioactive isotopes including, but not limited to, ¹⁸F with a half-life of approximately 110 minutes, ¹¹C with a half-life of approximately twenty minutes, ¹³N with a half-life of approximately ten minutes and ¹⁵O with a half-life of approximately two minutes, using the coincidence method. Therefore, for use as a PET agent dendrimers can be conjugated or complexed with one or more of the various positron emitting metal ions, such as ⁵¹Mn, ⁵²Fe, ⁶⁰Cu, ⁶⁸Ga, ⁷²As, ^{94m}Tc, or ¹¹⁰In. The disclosed dendrimers can also be conjugated or complexed with radionuclides such as ¹⁸F, ¹²⁴I, ¹²⁵I, ¹³¹I, ¹²³I, ⁷⁷Br, and ⁷⁶Br. Examples of metal radionuclides for scintigraphy or radiotherapy include ^{99m}Tc, ⁵¹Cr, ⁶⁷Ga, ⁶⁸Ga, ⁴⁷Sc, ⁵¹Cr, ¹⁶⁷Tm, ⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh ¹⁰⁹Pd, ^{117m}Sn, ¹⁴⁹Pm ¹⁶¹Tb, ¹⁷⁷Lu, ²²⁵Ac, ¹⁹⁸Au and ¹⁹⁹Au. The choice of metal will be determined based on the desired therapeutic or diagnostic application. For example, for diagnostic purposes, examples of radionuclides include ⁶⁴Cu, ⁶⁷Ga, ⁶⁸G, ^{99m}Tc, and ¹¹¹In. For therapeutic purposes, examples of radionuclides include ⁶⁴Cu, ⁹⁰Y ¹⁰⁵R, ¹¹¹In, ^{117m}Sn, ¹⁴⁹Pm ¹⁵³Sm, ¹⁶¹Tb, ¹⁶⁶Tb, ¹⁶⁶Dy, ¹⁶⁶Ho, ¹⁷⁵Yb, ¹⁷⁷Lu, ²²⁵Ac, ^{186/188}Re, and ¹⁹⁹Au. ^{99m}Tc is useful for diagnostic applications because of its low cost, availability, imaging properties, and high specific activity. The nuclear and radioactive properties of ^{99m}Tc make this isotope an ideal scintigraphic imaging agent. This isotope has a single photon energy of ¹⁴⁰ keV and a radioactive half-life of about ⁶ hours, and is readily available from a ⁹⁹Mo-^{99m}Tc generator. ¹⁸F, ⁴-[¹⁸F]fluorobenzaldehyde (¹⁸FB), Al[¹⁸F]-NOTA, ⁶⁸Ga-DOTA, and ⁶⁸Ga-NOTA are typical radionuclides for conjugation to dendrimers for PET imaging. ¹⁵³Sm can be used with chelators such as ethylenediaminetetramethylenephosphonic acid (EDTMP) chelator or 1,4,7,10-tetraazacyclododecanetetramethylenephosphonic acid (DOTMP).

Magnetic resonance imaging (MRI) is today widely used to assess brain disease, spinal disorder, angiography, cardiac function, and musculoskeletal damage. Although MRI requires a larger acquisition time than computed tomography (CT), MRI does not require the use of ionizing radiation and scans can be performed at any chosen orientation. It features full three-dimensional (3-D) capabilities, excellent soft-tissue contrast and high spatial resolution. Furthermore, MRI is very adept at morphological imaging and functional imaging. Thus, compositions of dendrimers conjugated or complexed with one or more imaging probes for magnetic resonance imaging including one or more MRI contrast agents are provided. Exemplary MRI contrast agents that can be delivered include Gd, Mn, BaSO₄, iron oxides, iron platinum.

### Dendrimer Conjugation to Imaging Agents Via Ether Linkages

Compositions including a hydroxyl-terminated dendrimer conjugated to a radionuclide or an MRI contrast agent via an ether linkage, optionally with one or more linkers/spacers are described. In one embodiment, ¹⁸F is conjugated onto a hydroxyl-terminated generation 4 PAMAM dendrimer as shown as compound 5 in FIG. 1. In another embodiment, ⁸⁹Zr is complexed into a hydroxyl-terminated generation 4 PAMAM dendrimer via chelation through p-SCN-Bn-Deferoxamine (DFO) that is conjugated to the dendrimer as shown as compound 5 in FIG. 3.

In some embodiments, the covalent bonds between the surface groups of the dendrimers and the linkers, or the dendrimers and the radionuclides (if conjugated without any linking moieties) are stable under in vivo conditions, i.e., minimally cleavable when administered to a subject and/or excreted intact from the body. For example, less than 5%, 4%, 3%, 2%, 1%, 0.5%, 0.4%, 0.3%, 0.2%, 0.1%, or less than 0.1% of the total dendrimer complexes have active agent such as radionuclides cleaved off without 24 hours, 48 hours, or 72 hours after in vivo administration. In some embodiments, these covalent bonds are ether bonds. In some embodiments, the covalent bond between the surface groups of the dendrimers and the linkers, or the dendrimers and the radionuclides (if conjugated without any linking moieties) are not hydrolytically or enzymatically cleavable bonds such as ester bonds.

In some embodiments, one or more hydroxyl groups of hydroxyl-terminated dendrimers conjugate to one or more linking moieties and one or more radionuclides via one or more ether bonds as shown in Formula (I) below.

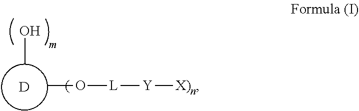

wherein D is a dendrimer (e.g., a G2 to G10 dendrimer, such as a G2 to G10 poly(amidoamine) (PAMAM) dendrimer); L is one or more linking moieties or spacers; X is a radionuclide or a chelator for complexing with a radionuclide or an MRI contrast agent; n is an integer from 1 to 100; and m is an integer from 16 to 4096;
and Y is a linker selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, thioether (—S—, —SR—), and ethers (—O—, —OR—), wherein R is an alkyl group, an aryl group, or a heterocyclic group. In some embodiments, Y is a bond or linkage that is minimally cleavable in vivo. In some embodiments, Y includes a C₁-C₁₂, such as a C₁-C₆ alkyl group, or a polyethylene glycol linker.

In one embodiment, D is a G4 PAMAM dendrimer; L is one or more linking or spacer moieties; R is ¹⁸F; n is about 8-12; m is an integer about 52-56; and where n+m=64. In some embodiments, L includes a C₁-C₁₂ alkyl group, such as a C₁-C₆ alkyl group, or a polyethylene glycol linker.

In another embodiment, D is a G4 PAMAM dendrimer; L is one or more linking or spacer moieties; R is p-SCN-Bn-Deferoxamine; n is about 8-12; m is an integer about 52-56; and where n+m=64.

i. Chelation Agents

In some embodiments, one or more radionuclides is conjugated with the dendrimer via a chelating agent. Metal radionuclides can be chelated by, for example, linear, macrocyclic, terpyridine, and N₃S, N₂S₂, or N₄ chelants (see also, U.S. Pat. Nos. 5,367,080, 5,364,613, 5,021,556, 5,075,099, 5,886,142), and other chelators known in the art including, but not limited to, HYNIC, DTPA, EDTA, DOTA, DO3A, TETA, NOTA and bisamino bisthiol (BAT) chelators (see also U.S. Pat. No. 5,720,934). For example, N₄ chelators are described in U.S. Pat. Nos. 6,143,274; 6,093,382; 5,608,110; 5,665,329; 5,656,254; and 5,688,487. Certain N₃₅ chelators are described in PCT/CA94/00395, PCT/CA94/00479, PCT/CA95/00249 and in U.S. Pat. Nos. 5,662,885; 5,976,495; and 5,780,006. The chelator also can include derivatives of the chelating ligand mercapto-acetyl-acetyl-glycyl-glycine (MAG3), which contains an N₃S, and N₂S₂ systems such as MAMA (monoamidemonoaminedithiols), DADS (N₂S diaminedithiols), and CODADS. These ligand systems and a variety of others are described in, for example, Liu, S, and Edwards, D., 1999. Chem. Rev., 99:2235-2268, and references therein.

The chelator also can include complexes containing ligand atoms that are not donated to the metal in a tetradentate array. These include the boronic acid adducts of technetium and rhenium dioximes, such as are described in U.S. Pat. Nos. 5,183,653; 5,387,409; and 5,118,797, the disclosures of which are incorporated by reference herein, in their entirety.

In one embodiment, the chelator is p-SCN-Bn-Deferoxamine (DFO) for chelating ⁸⁹Zr. In another embodiment, the chelator is DOTA for chelating ⁹⁰Y and/or Gd.

The chelators can be covalently linked to the dendrimers, optionally via a linker, and then directly labeled with the radionuclides or MRI contrast agents. In some embodiments, the dendrimer is conjugated to the chelators with or without a linker through an ether linkage for enhanced stability. Dendrimers comprising ¹⁸F, ⁸⁹Zr (Zirconium-89) or Gd are provided herein for diagnostic imaging. Complexes of radioactive technetium are also useful for diagnostic imaging, and complexes of radioactive rhenium are particularly useful for radiotherapy.

ii. Fluorine-18/Dendrimer Conjugates

In some embodiments, dendrimers are conjugated to ¹⁸F (Fluorine-18). ¹⁸F (Fluorine-18) is the most commonly used isotope for PET imaging. It is a fluorine isotope with high positron decay ratio, low energy, favorable half-life (109.8 minutes), and high specific activity and well-established radiochemistry. It decays by emitting positrons having the lowest positron energy which contributes to a high-resolution imaging acquire. Multiple ¹⁸F radiotracers were approved by the FDA for clinical applications, for example, 2-deoxy-2-¹⁸F-fluoro-β-D-glucose (¹⁸F-FDG), which is an analogue of glucose, is used for the early detection of tumors. Thus, in some embodiments, the radionuclide to be conjugated to hydroxyl-terminated dendrimers via ether linkage is ¹⁸F. An exemplary hydroxyl-terminated dendrimers via ether linkage conjugated to ¹⁸F is shown in FIG. 1.

iii. Zirconium-89 Dendrimer Conjugates

In some embodiments, dendrimers are conjugated to ⁸⁹Zr (Zirconium-89). ⁸⁹Zr is another PET isotope with long half-life (78.4 hours) which matches pharmacokinetics of antibodies and has relative low average positron energy of 395 keV.19. ⁸⁹Zr-based radiotracers are safer to handle and are more stable in vivo making good candidates for clinical applications. Thus, in some embodiments, the radionuclide to be conjugated to hydroxyl-terminated dendrimers via ether linkage is ⁸⁹Zr. An exemplary hydroxyl-terminated dendrimers via ether linkage conjugated to ⁸⁹Zr is shown in FIG. 3.

iv. Yttrium-90 Dendrimer Conjugates

In some embodiments, dendrimers are conjugated to ⁹⁰Y (Yttrium-90). ⁹⁰Y is an isotope of yttrium. ⁹⁰Y is a pure beta-emitter with average decaying energy of 0.93 MeV and the average penetration depth in human tissue is 4-5 mm. The physical half-life of Y-90 is 64.2 h. Yttrium-90 has found a wide range of uses in radiation therapy to treat some forms of cancer.

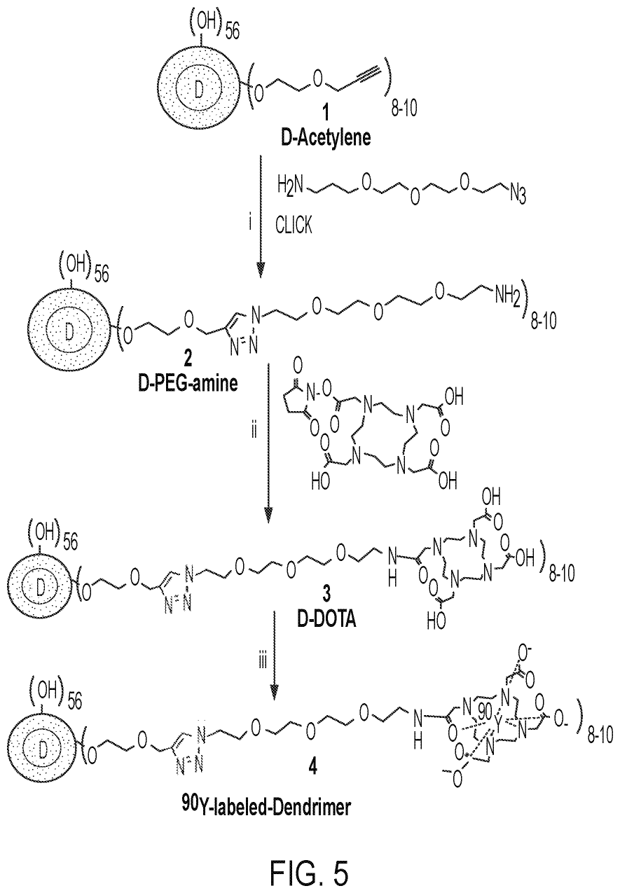

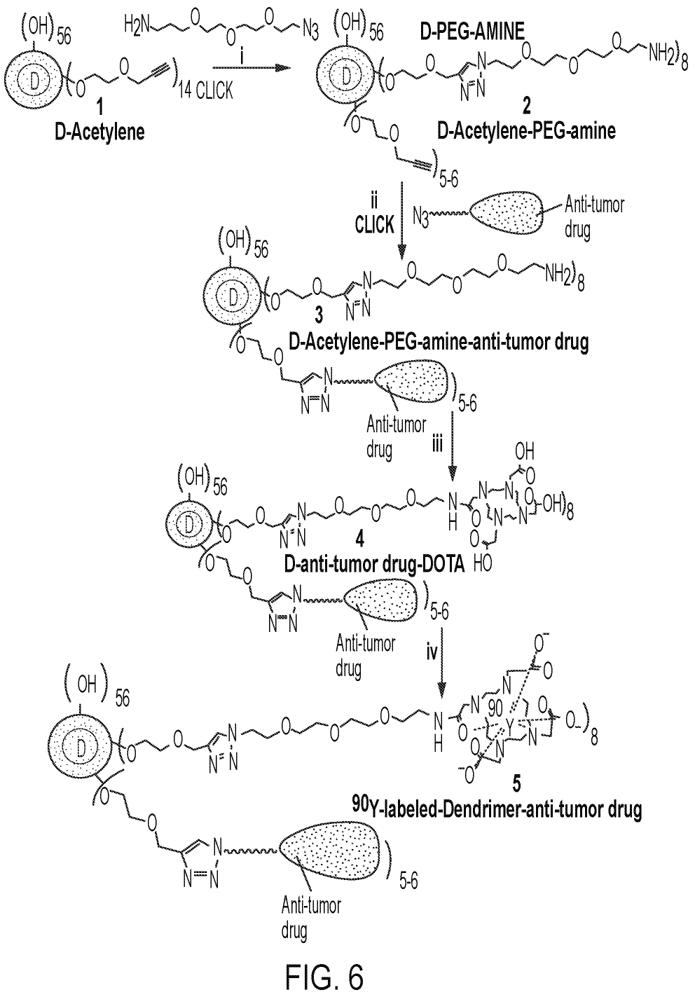

Thus, in some embodiments, the radionuclide to be conjugated to hydroxyl-terminated dendrimers via ether linkage is ⁹⁰Y. An exemplary hydroxyl-terminated dendrimers via ether linkage conjugated to DOTA and chelated with ⁹⁰Y is shown in FIG. 5. Alternatively, Gd may be chelated with this hydroxyl-terminated dendrimer DOTA conjugate. For cancer treatment, dendrimers labeled with one or more radionuclides can be further conjugated to one or more anti-cancer drugs. An exemplary structure of a dendrimer labeled with one or more radionuclides that are further conjugated to one or more anti-cancer drugs is shown in FIG. 6.

### Spacers and Linking Agents

In some embodiments, the attachment of an imaging or diagnostic agent to the dendrimer occurs via one or more of disulfide, ether, thioester, carbamate, carbonate, hydrazine, or amide linkages. In some embodiments, the attachment occurs via an appropriate spacer that provides an ether bond or an amide bond between the radionuclide and the dendrimer, depending on the desired release kinetics of the active agent. The one or more spacers/linkers between a dendrimer and a radionuclide can be designed to provide a non-releasable or minimally releasable form of the dendrimer/agent complexes in vivo. In some embodiments, an ether bond is introduced for non-releasable, or minimally releasable form of dendrimer complexes. In addition, one or more organic functional groups can be chosen to facilitate the covalent attachment of the active agents to the dendrimer/agent conjugates.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, —CHRO₂C—), wherein R is an alkyl group, an aryl group, or a heterocyclic group.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is between 3 and 200 atoms, between 3 and 150 atoms, between 3 and 100 atoms, or between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the radionuclide and the dendrimer. In some embodiments, the spacer is polyethylene glycol.

Compositions and methods for conjugating agents with dendrimers are known in the art, and are described in detail in U.S. Published Application Nos. US 2011/0034422, US 2012/0003155, and US 2013/0136697. In some embodiments, the active agents are functionalized for conjugation to the dendrimer/agent conjugate, optionally via one or more linking moieties. In some embodiments, the functionalized active agents and/or linking moieties are designed to have desirable release rate of the active agents from the dendrimer/agent conjugates in vivo. The functionalized active agents and/or linking moieties can be designed to be cleaved hydrolytically, enzymatically, or combinations thereof, to provide for the sustained release of the active agents in vivo. In some embodiments, the functionalized active agents and/or linking moieties are designed to remain bound to the dendrimer/agent conjugates in vivo.

Therefore, in some embodiments, the functionalized active agents and/or linking moieties are designed to be cleaved at a minimal or insignificant rate in vivo. The composition of the linking moiety can also be selected in view of the desired release rate of the active agents. In some embodiments, one or more active agents are functionalized to be non-cleavable or minimally cleavable from the dendrimer/agent conjugates in vivo, for example via ether linkage, optionally, with one or more spacers/linkers.

The optimal loading of imaging agents such as radionuclides will necessarily depend on many factors, including the choice of radionuclide, dendrimer structure and size, and tissues to be targeted/imaged. In some embodiments, the one or more imaging agents are encapsulated, associated, and/or conjugated to the dendrimer at a concentration of about 0.01% to about 45%, about 0.1% to about 30%, about 0.1% to about 20%, about 0.1% to about 10%, about 1% to about 10%, about 1% to about 5%, about 3% to about 20% by weight, and about 3% to about 10% by weight. However, optimal loading for any imaging agents, dendrimer, and site of target can be identified by routine methods, such as those described.

In some embodiments, conjugation of imaging agents and/or linkers occurs through one or more surface and/or interior groups. Thus, in some embodiments, the conjugation of active agents/linkers occurs via about 1%, 2%, 3%, 4%, or 5% of the total available surface functional groups, such as hydroxyl groups, of the dendrimer/agent prior to the conjugation. In other embodiments, the conjugation of active agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimer prior to the conjugation. In some embodiments, dendrimer/agent complexes retain an effective amount of surface functional groups for targeting to specific cell types, whilst conjugated to an effective amount of active agents for treat, prevent, and/or image the disease or disorder.

In some aspects, the disclosure provides compounds comprising a dendrimer conjugated to one or more agents (e.g., imaging agent, therapeutic agents) through a terminal ester, ether, or amide bond. In some embodiments, the dendrimer comprises surface (e.g., terminal) hydroxyl groups optionally substituted with the agents. As used herein, in some embodiments, a dendrimer conjugate refers to a compound comprising a dendrimer conjugated to one or more agents. In some embodiments, the dendrimer conjugate comprises a dendrimer conjugated to an imaging agent. In some embodiments, the dendrimer conjugate comprises a dendrimer conjugated to an imaging agent or a therapeutic agent. In some embodiments, the dendrimer conjugate comprises a dendrimer conjugated to an imaging agent and a therapeutic agent.

In some aspects, the disclosure provides a composition comprising a compound that comprises a dendrimer conjugated to an agent (e.g., an imaging agent and/or a therapeutic agent) through a terminal ester, ether, or amide bond. In some embodiments, the dendrimer comprises a high-density of terminal hydroxyl groups optionally substituted with the agent. In some embodiments, a compound comprising a dendrimer conjugated to an agent is 10-20% by mass of agent. In some embodiments, the terminal ester, ether, or amide bond is conjugated to the agent through a linker or a spacer. In some embodiments, the dendrimer is conjugated to the agent through a terminal ether bond. In some embodiments, the compound is about 10% to about 15% by mass of agent. In some embodiments, the compound is about 15% to about 20% by mass of agent. In some embodiments, at least 50% of terminal sites on the dendrimer comprise terminal hydroxyl groups. In some embodiments, at least 50% and up to 99% (e.g., 50-95%, 50-90%, 50-80%, 50-70%, 50-60%, 60-80%, 70-90%) of terminal sites on the dendrimer comprise terminal hydroxyl groups.

In some embodiments, an agent (e.g., an imaging agent or a therapeutic agent) of a compound described herein has an aqueous solubility that is increased relative to an unconjugated compound comprising the agent in absence of dendrimer. In some embodiments, the aqueous solubility is increased by at least 10% relative to the unconjugated compound. In some embodiments, the aqueous solubility is increased by between about 10% and about 100% relative to the unconjugated compound. In some embodiments, the aqueous solubility is increased by at least about a factor of two relative to the unconjugated compound. In some embodiments, the aqueous solubility is increased by between about a factor of two and about a factor of ten relative to the unconjugated compound. In some embodiments, the aqueous solubility is solubility under physiological conditions. In some embodiments, the aqueous solubility is solubility in water having a pH of between about 7.0 and about 8.0. In some embodiments, the imaging agent is present at a concentration at which the unconjugated compound is insoluble under physiological conditions.

In some embodiments, surface functional groups (e.g., terminal functional groups) of a dendrimer include one or more hydroxyl groups, one or more amine groups, and/or one or more carboxyl groups. In some embodiments, the terminal functional groups of a dendrimer provide attachment sites through which the at least one agent (e.g., an imaging agent and/or a therapeutic agent) is conjugated to form a dendrimer conjugate. Accordingly, in some embodiments, the at least one agent is conjugated to the dendrimer through an ether bond, an amide bond, or an ester bond formed by conjugation to a terminal functional group of the dendrimer. In some embodiments, the at least one agent is conjugated to the dendrimer through an ether bond or an amide bond. In some embodiments, the at least one agent is conjugated to the dendrimer through an ether bond.

In some embodiments, the number of terminal sites on a dendrimer can depend on the particular dendrimeric scaffold and its generation. For example, in some embodiments, a dendrimer is based on a generation 0, 1, 2, 3, 4, 5, 6, 7, 8, 9, or 10 PAMAM dendrimeric scaffold, which have 4, 8, 16, 32, 64, 128, 256, 512, 1024, 2048, and 4096 terminal sites, respectively. However, it should be appreciated that different dendrimeric scaffolds having a different number of terminal sites at each generation can be used in accordance with the disclosure.

In some embodiments, all terminal sites of a dendrimer comprise hydroxyl groups. In some embodiments, each terminal site of a dendrimer comprises either a hydroxyl group or an amine group. In some embodiments, each terminal site of a dendrimer conjugate comprises a hydroxyl group, an amine group, or an agent (e.g., an imaging agent or a therapeutic agent) conjugated to the dendrimer through an ether or amide bond. In some embodiments, each terminal site of a dendrimer conjugate comprises either a hydroxyl group or an agent conjugated to the dendrimer through an ether bond.

In some embodiments, at least 50% of terminal sites on a dendrimer conjugate comprise hydroxyl groups (e.g., at least 50% of terminal sites do not comprise either an amine group or an agent). For example, in some embodiments, at least 60%, at least 70%, at least 80%, at least 90%, at least 95%, at least 98%, or at least 99% of terminal sites on a dendrimer conjugate comprise hydroxyl groups. In some embodiments, about 50-99%, about 60-99%, about 70-99%, about 80-99%, about 90-99%, about 95-99%, about 98-99%, about 70-95%, about 70-90%, about 80-95%, or about 80-90% of terminal sites on a dendrimer conjugate comprise hydroxyl groups.

In some embodiments, one or more terminal sites on a dendrimer conjugate comprise an agent (e.g., an imaging agent and/or a therapeutic agent). In some embodiments, at least 2, at least 3, at least 4, at least 5, at least 10, at least 15, at least 20, or more, terminal sites on a dendrimer conjugate comprise an agent. In some embodiments, at least 1% of terminal sites on a dendrimer conjugate comprise an agent. For example, in some embodiments, at least 2%, at least 5%, at least 10%, at least 15%, at least 20%, at least 25%, or at least 30% of terminal sites on a dendrimer conjugate comprise an agent. In some embodiments, about 1-50%, about 1-40%, about 1-25%, about 1-10%, about 5-50%, about 5-40%, about 5-25%, about 5-10%, about 10-50%, about 10-40%, or about 10-25% of terminal sites on a dendrimer conjugate comprise an agent. In some embodiments, about 1%, about 2%, about 3%, about 4%, or about 5% of terminal sites on a dendrimer comprise an agent. In some embodiments, less than 5%, less than 10%, less than 15%, less than 20%, less than 25%, less than 30%, less than 35%, less than 40%, less than 45%, less than 50%, less than 55%, less than 60%, less than 65%, less than 70%, less than 75% of terminal sites on a dendrimer comprise an agent. In some embodiments, a dendrimer conjugate has an effective amount of terminal functional groups (e.g., terminal hydroxyl groups) for targeting to a specific cell type, while having to an effective amount of agent for imaging and/or treating as described herein. In some embodiments, terminal sites of a dendrimer conjugate can be evaluated using proton nuclear magnetic resonance (¹H NM/R), or other analytical methods known in the art, to determine a percentage of terminal sites having an agent and/or terminal functional group.

In some embodiments, a desired agent loading can depend on certain factors, including the choice of agent, dendrimer structure and size, and cell or tissue to be treated. In some embodiments, a dendrimer conjugate is about 0.01% to about 45% by mass (m/m) of agent (e.g., imaging agent and/or therapeutic agent). In some embodiments, a dendrimer conjugate is about 10% to about 20% by mass of agent. In some embodiments, a dendrimer conjugate is about 0.1% to about 30%, about 0.1% to about 20%, about 0.1% to about 10%, about 1% to about 10%, about 1% to about 5%, about 3% to about 20%, about 3% to about 10% by mass of agent.

As described herein, in some embodiments, a dendrimer conjugate can be characterized in terms of mass percentage (e.g., % by mass (m/m)) of agent. In some embodiments, mass percentage refers to a molecular weight (Da) percentage of agent in a dendrimer conjugate. In some embodiments, mass percentage can be determined by the general formula of: (agent Mw)/(conjugate Mw)×100. For example, in some embodiments, (agent Mw) can be determined by calculating or approximating the molecular weight of an agent as a single molecule or compound (conjugated or unconjugated), and multiplying this value by the number of terminal sites at which the agent is present in a dendrimer conjugate. In some embodiments, (agent Mw) can be determined by calculating or approximating the sum of the atomic mass of all atoms which form the agent in a dendrimer conjugate. The value for (agent Mw) can be taken as a fraction of total molecular weight of the dendrimer conjugate (conjugate Mw), and multiplied by 100 to provide a mass percentage. In some embodiments, mass percentage can be determined by experimental or empirical means. For example, in some embodiments, mass percentage can be determined using proton nuclear magnetic resonance (¹H NMR) or other analytical methods known in the art.

In some embodiments, the attachment of dendrimer to agent occurs via an appropriate spacer that provides an ether bond between the agent and the dendrimer/agent. In some embodiments, one or more spacers/linkers are added between a dendrimer and an active agent to achieve desired and effective binding and/or pharmacokinetics in vivo.

The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide. The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl and thiol terminations.

### Formulations for Nuclear Imaging (Radionuclide Imaging) and Radiotherapy

In some embodiments, dendrimers conjugated to one or more diagnostic or imaging agents including radionuclides are formulated for use in nuclear imaging (Radionuclide Imaging) and radiotherapy techniques. In some embodiments, the unit dose to be administered has a radioactivity of about 0.01 mCi to about 100 mCi, or about 1 mCi to about 20 mCi. In some embodiments, the solution to be injected at unit dosage is from about 0.01 mL to about 10 mL. In some embodiments, dendrimer conjugates can be formed as radioactive complexes in solutions containing radioactivity at concentrations of from about 0.01 mCi to 100 mCi per mL.

In some embodiments, doses of a radionuclide-labeled dendrimers can provide 10-20 mCi. In some embodiments, after injection of the radionuclide-labeled dendrimers into the patient, a gamma camera calibrated for the gamma ray energy of the nuclide incorporated in the imaging agent is used to image areas of uptake of the agent and quantify the amount of radioactivity present in the site. Imaging of the site in vivo can take place in a matter of a few minutes. However, imaging can take place, if desired, in hours or even longer, after the radiolabeled dendrimer composition is administered into a patient. In some embodiments, a sufficient amount of the administered dose will accumulate in the area to be imaged within about 0.1 of an hour to permit the taking of scintiphotos.

Proper dose schedules for the disclosed radiotherapeutic compounds are known to those skilled in the art. The compounds can be administered using many methods including, but not limited to, a single or multiple IV or IP injections, using a quantity of radioactivity that is sufficient to cause damage or ablation of the targeted tissue, but not so much that substantive damage is caused to non-target (normal tissue). The quantity and dose required is different for different constructs, depending on the energy and half-life of the isotope used, the degree of uptake and clearance of the agent from the body and the mass of the target tissue. In general, doses can range from a single dose of about 30-50 mCi to a cumulative dose of up to about 3 Ci.

The radiotherapeutic compositions can include physiologically acceptable buffers, and can require radiation stabilizers to prevent radiolytic damage to the compound prior to injection. Radiation stabilizers are known to those skilled in the art, and can include, for example, para-aminobenzoic acid, ascorbic acid, gentisic acid and the like.

### Additional Active Agents

In some embodiments, dendrimers complexed or conjugated with one or more radionuclides are further conjugated with one or more active agents. Exemplary additional active agents include therapeutic, prophylactic or diagnostic agents.

Dendrimers have the advantage that multiple therapeutic, prophylactic, and/or diagnostic agents can be delivered with the same dendrimers. Therefore, one or more types of active agents can be encapsulated, complexed or conjugated to the dendrimer/imaging agent. In one embodiment, the dendrimer/imaging agent are complexed with or conjugated to two or more different classes of agents, providing simultaneous delivery with different or independent release kinetics at the target site. Agents to be included can be proteins or peptides, sugars or carbohydrate, nucleic acids or oligonucleotides, lipids, small molecules (e.g., molecular weight less than 2,500 Daltons, less than 2,000 Daltons, or less than 1,500 Daltons). The nucleic acid can be an oligonucleotide encoding a protein, for example, a DNA expression cassette or an mRNA. Representative oligonucleotides include siRNAs, microRNAs, DNA, and RNA. In some embodiments, the additional active agent is a therapeutic antibody.

For example, in some embodiments, dendrimer/imaging agent are covalently linked to at least one additional detectable moiety and/or at least one other class of agents. In a further embodiment, dendrimer/imaging agent complexes each carrying different classes of additional agents are administered simultaneously for a combination treatment.

The selective targeting of dendrimer allows less active agent to be administered to achieve the same therapeutic effect compared to the same active agent without conjugating to dendrimer, thus, reducing dose-related cytotoxicity and/or other side effects side effects associated with the active agent. The dendrimer can also increase solubility of the one or more additional therapeutic, prophylactic, and/or diagnostic agents to be delivered. Examples of additional active agents include therapeutic agents that have been shown to have efficacy for treating and preventing one or more inflammatory diseases or disorders or cancers.

In some embodiments, an additional active agent is a diagnostic agent. Useful additional diagnostic agents include moieties that can be administered in vivo and subsequently detected. The additional agent can be, for example, any moiety that facilitates detection, either directly or indirectly, by a non-invasive and/or in vivo visualization technique. Examples of additional diagnostic agents include paramagnetic molecules, fluorescent compounds, magnetic molecules, x-ray imaging agents, and contrast media. Examples of other suitable contrast agents include gases or gas emitting compounds, which are radiopaque. In some embodiments, the dendrimer complexes further include agents useful for determining the location of administered compositions, such as fluorescent tags, and contrast agents. Exemplary diagnostic agents include dyes, fluorescent dyes, near infra-red dyes, fluorescent molecules (a.k.a. fluorochromes and fluorophores), chemiluminescent reagents (e.g., luminol), bioluminescent reagents (e.g., luciferin and green fluorescent protein (GFP)), metals (e.g., gold nanoparticles), and radioactive isotopes (radioisotopes). Suitable detectable labels can be selected based on the choice of imaging method. For example, in some embodiments, the additional detectable label is a near infrared fluorescent dye for optical imaging, a Gadolinium chelate for MRI imaging, or a gold nanoparticle for CT imaging.

In some embodiments, an additional agent is both an imaging agent and a diagnostic agent, and/or a therapeutic agent. In some embodiments, the additional agent is a tantalum compound, an organic iodo acid, such as iodo carboxylic acid, triiodophenol, iodoform, and/or tetraiodoethylene, a non-radioactive detectable agent, e.g., a non-radioactive isotope, such as iron oxide and Gd, an enzyme, fluorophores, and quantum dots (QDOT®), Lissamine Rhodamine PE, a fluorescent or non-fluorescent stain or dye, for example, that can impart a visible color or that reflects a characteristic spectrum of electromagnetic radiation at visible or other wavelengths, for example, infrared or ultraviolet, such as Rhodamine, a ferromagnetic compound, a paramagnetic compound, such as gadolinium, a superparamagnetic compound, such as iron oxide, and a diamagnetic compound, such as barium sulfate.

In some embodiments, one or more additional active agents are functionalized, for example with ether or amide linkages, optionally, with one or more spacers/linkers, for ease of conjugation with the dendrimer for desired release kinetics. In some embodiments, the one or more additional active agents are functionalized to be non-cleavable or minimally cleavable from the dendrimer conjugates in vivo, for example via ether optionally with one or more spacers/linkers. In other embodiments, the one or more additional active agents delivered via dendrimer conjugates are released from the dendrimer complexes after administration to a mammalian subject in an amount effective to be therapeutically effective at the target cells, tissues, regions for at least 1 day, 2 days, 3 days, 4 days, 5 days, 6 days, at least a week, 2 weeks, or 3 weeks, at least a month, two months, three months, four months, five months, or six months.

Agents and/or targeting moiety can be either covalently attached or intra-molecularly dispersed or encapsulated in dendrimer conjugates. In some embodiments, the dendrimer conjugated to one or more radionuclides or MRI contrast agents is a dendrimer, such as a PAMAM dendrimer, up to generation 10 having hydroxyl terminations.

### Methods for Making Dendrimers/Agents Complexes

In some aspects, the disclosure provides methods of making dendrimers complexed or conjugated with one or more imaging or diagnostic agents. The methods conjugate dendrimers with one or more radionuclide reporters appropriate for scintigraphy, SPECT, or PET imaging, one or more MRI contrast agents, and/or with one or more radionuclides appropriate for radiotherapy. Methods of making dendrimer complexes in which the dendrimers are conjugated with a first radionuclide useful for diagnostic imaging and a second radionuclide useful for radiotherapy are specifically contemplated, and in some embodiments, the conjugation to the first and/or second radionuclide (or the appropriate chelator of the first and/or second radionuclide) is via ether linkages. Therefore, in some embodiments, a singular dendrimer/radionuclide composition can simultaneously treat and/or diagnose a disease or a condition at one or more locations in the body. Also disclosed are radioactively labeled SPECT or scintigraphic imaging agents that have a suitable amount of radioactivity.

### Methods for Making Dendrimers

Dendrimers can be purchased or prepared via a variety of chemical reaction steps. Dendrimers are usually synthesized according to methods allowing controlling their structure at every stage of construction. The dendritic structures are mostly synthesized by two main different approaches: divergent or convergent.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. In some embodiments, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of agents or moieties bound thereto, directly or indirectly through a linker.

In some embodiments, dendrimers are prepared using different methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward building inward and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of active agents are linked to one type of dendron and a different type of active agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO 2009/046446, WO 2015168347, WO 2016025745, WO 2016025741, WO 2019094952, and U.S. Pat. No. 8,889,101.

Dendrimer Complexes with Radionuclides and/or NRI Contrast Agents

Methods of making dendrimer complexes with one or more radionuclides or MRI contrast agents are described. Methods for conjugating agents to dendrimers are known in the art, and are described in detail in U.S. Published Application Nos. US 2011/0034422, US 2012/0003155, and US 2013/0136697.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given active agent can be selected in view of the linking moiety desired, as well as the structure of the agents and dendrimerd as a whole as it relates to compatibility of functional groups, protecting group strategies, and the presence of labile bonds.

### Dendrimer Conjugation to Radionuclides or NRI Contrast Agents via Ether Linkages

Also provided is a method to incorporate a radionuclide and/or an MRI contrast agent onto a hydroxyl-terminated dendrimer via an ether linkage, optionally via one or more linkers/spacers.

In some embodiments, surface or terminal groups of hydroxyl-terminated dendrimers are modified via etherification reaction prior to conjugation to one or more linkers/spacers and one or more active agents (e.g., radionuclides or MRI contrast agents). Etherification is the dehydration of an alcohol to form ethers. In some embodiments, one or more hydroxyl groups of hydroxyl-terminated dendrimers undergo etherification reaction prior to conjugation to one or more linking moieties and one or more active agents.

In one embodiment, ether linkage is introduced at the surface groups of hydroxyl PAMAM dendrimer by reacting with propargyl bromide in the presence of 2% sodium hydroxide solution in DMSO as described in Example 1 (FIG. 1). In a further embodiment, etherification reaction of generation 4 hydroxyl-terminated PAMAM dendrimer, PAMAM-G4—OH, using allyl bromide, anhydrous cesium carbonate and tetrabutylammonium iodide in DMF is described in Example 3 and the reaction scheme is shown in FIG. 3.

Exemplary synthetic routes are demonstrated in FIGS. 1 and 3. ¹⁸F is conjugated onto a hydroxyl-terminated generation 4 PAMAM dendrimer as shown as compound 5 in FIG. 1; and ⁸⁹Zr is complexed into a hydroxyl-terminated generation 4 PAMAM dendrimer via chelation through p-SCN-Bn-Deferoxamine (DFO) that is conjugated to the dendrimer as shown as compound 5 in FIG. 3.

### Pharmaceutical Formulations

Pharmaceutical compositions including dendrimer conjugated to one or more radionuclide and/or one or more MRI contrast agents, and optionally one or more active agents, may be formulated in a conventional manner using one or more physiologically acceptable carriers including excipients and auxiliaries which facilitate processing of the active compounds into preparations which can be used pharmaceutically. Proper formulation is dependent upon the route of administration chosen. In some embodiments, the compositions are formulated for parenteral delivery. In some embodiments, the compositions are formulated for subcutaneous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more dendrimer/agent complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof.

Suitable pharmaceutically acceptable excipients are, in some embodiments, selected from materials which are generally recognized as safe (GRAS), and may be administered to an individual without causing undesirable biological side effects or unwanted interactions. See, for example, Remington's Pharmaceutical Sciences, 20th ed., Lippincott Williams & Wilkins, Baltimore, MD, 2000, p. 704.

In some embodiments, the compositions are formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

Pharmaceutical compositions formulated for administration by parenteral (intramuscular, intraperitoneal, intravenous or subcutaneous injection) and enteral routes of administration are described.

### Parenteral Administration

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion. The dendrimer compositions can be administered parenterally, for example, by subdural, intravenous, intrathecal, intraventricular, intraarterial, intra-amniotic, intraperitoneal, or subcutaneous routes. In some embodiments, the dendrimer compositions are administered via subcutaneous injection.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimer compositions can also be administered in an emulsion, for example, water in oil. Examples of oils are those of petroleum, animal, vegetable, or synthetic origin, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are examples of liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### Enteral Administration

The compositions can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are examples of liquid carriers. These can also be formulated with proteins, fats, saccharides and other components of infant formulas.

In some embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are provided in certain embodiments. Elixirs and syrups also are well known oral formulations.

### Methods of Use

The compositions of dendrimers conjugated to one or more radionuclides or one or more MRI imaging agents are suitable for several applications, including but not limited to diagnostic, therapeutic and analytical applications. In particular, disclosed are methods of identifying and labeling one or more sites of inflammation or cancer in a subject by administering to the subject the dendrimer composition.

Macrophages (Mφ) play many important roles in the immune responses of infected tissues through a polarized activation phase. Activated macrophages (Mφ*) are mainly classified as M1 (pro-inflammation) and M2 (anti-inflammation) macrophages. Both M1 and M2 macrophages have important roles for the inflammatory processes of phagocytosis, antigen presentation, and scavenging activities (M1), as well as for the processes of wound-healing and tumor growth (M2). However, direct targeting of select population of macrophages, i.e., to discriminate between non-activated (Mφ) and activated macrophages (Mφ*) is challenging.

Compositions of dendrimers conjugated to one or more radionuclides or one or more MRI imaging agents are suitable for diagnosing and imaging a specific state or condition in a subject, e.g., one or more inflammatory diseases such as in rheumatoid arthritis, neuroinflammation such as cerebral palsy, neurodegenerative disorders such as Alzheimer's disease and ALS, and cancer in vivo in a subject in need thereof. In particular, the disclosed methods and compositions are suitable for diagnosing, imaging, and/or treating one or more diseases or conditions associated with activated macrophages including inflammatory diseases such as Alzheimer's disease, ALS, dementia, hepatitis, atherosclerosis, rheumatoid arthritis, and cancer.

### Detecting and Imaging Regions of Inflammation

Methods of imaging inflammation using dendrimers conjugated to one or more radionuclides or one or more MRI imaging agents are described. Methods include administering an effective amount of dendrimer conjugates to a subject having a disease or disorder associated with inflammation and/or cancer. In some embodiments, dendrimers are conjugated to one or more radionuclides or one or more MRI imaging agents via ether linkages for enhanced stability in vivo.

In some embodiments, the methods are suitable for imaging one or more regions of inflammation associated with systemic viral and/or bacterial infections, systemic inflammatory response syndrome, sepsis, or septic shock. In some embodiments, the methods are suitable for imaging one or more regions of inflammation caused by any mechanism of macrophage activation including macrophage activation syndrome. In some embodiments, the methods are suitable for imaging one or more regions of inflammation associated with multi-organ dysfunction including neuroinflammation. In some embodiments, the methods are suitable for imaging one or more regions of inflammation associated with over-reactive M1 macrophages and/or elevations in proinflammatory markers such as IL-6, CRP, ferritin, and IL-lb. In some embodiments, the methods are suitable for imaging one or more regions of inflammation characterized by cytokine storm.

### Autoimmune or Inflammatory Disease

Methods of using dendrimers conjugated to one or more radionuclides or one or more MRI imaging agents for imaging and/or diagnosing inflammation associated with one or more autoimmune or inflammatory diseases or disorders are described. Exemplary autoimmune or inflammatory diseases or disorders include rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the inflammation is a chronic inflammation which is a prolonged, dysregulated and maladaptive response that involves active inflammation, tissue destruction and attempts at tissue repair. Persistent inflammation is associated with many chronic human conditions and diseases, including allergy, atherosclerosis, cancer, arthritis and autoimmune diseases.

### Neuroinflammation

Methods of using dendrimers conjugated to one or more radionuclides or one or more MRI imaging agents for imaging and/or diagnosing neuroinflammation are also described.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review ofPsychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, I L et al., Nat Rev Neurosci 2007, 8, 57). The delivery of active agents across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can be used to deliver imaging/diagnostic agents to the site of neuroinflammation associated with a neurological or neurodegenerative disease or disorder or central nervous system disorder. In some embodiments, the compositions and methods are effective in selectively delivering PET imaging probes to activated macrophages associated with neuroinflammation. For example, the compositions and methods can be used to image, diagnose, and/or treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

In some embodiments, methods and compositions of the disclosure can be used for imaging one or more sites of neuroinflammation in a subject, where the one or more sites of neuroinflammation are associated with a neurodegenerative disorder, such as Alzheimer's disease or ALS. In some embodiments, methods and compositions of the disclosure can be used for imaging and/or treating a neurodegenerative disorder, such as Alzheimer's disease or ALS. The inventors have demonstrated that a labeled dendrimer of the disclosure detected neuroinflammation in the cortex and hippocampus of mice with early stage Alzheimer's plaques, whereas a TSPO radiotracer was unable to detect neuroinflammation in these mice (Example 7). The improved sensitivity and selectivity of labeled dendrimer relative to TSPO radiotracer in models of neuroinflammation demonstrates the effectiveness of labeled dendrimers as imaging agents to detect sites of neuroinflammation associated with neurodegenerative disorder.

In some embodiments, the disorder is a peroxisomal disorder or leukodystrophy characterized by detrimental effects on the growth or maintenance of the myelin sheath that insulates nerve cells. The leukodystrophy can be, for example, 18q Syndrome with deficiency of myelin basic protein, Acute Disseminated Encephalomyeolitis (ADEM), Acute Disseminated Leukoencephalitis, Acute Hemorrhagic Leukoencephalopathy, X-Linked Adrenoleukodystrophy (ALD), Adrenomyeloneuropathy (AMN), Aicardi-Goutieres Syndrome, Alexander Disease, Adult-onset Autosomal Dominant Leukodystrophy (ADLD), Autosomal Dominant Diffuse Leukoencephalopathy with neuroaxonal spheroids (HDLS), Autosomal Dominant Late-Onset Leukoencephalopathy, Childhood Ataxia with diffuse CNS Hypomyelination (CACH or Vanishing White Matter Disease), Canavan Disease, Cerebral Autosomal Dominant Arteropathy with Subcortical Infarcts and Leukoencephalopathy (CADASIL), Cerebrotendinous Xanthomatosis (CTX), Craniometaphysical Dysplasia with Leukoencephalopathy, Cystic Leukoencephalopathy with RNASET2, Extensive Cerebral White Matter abnormality without clinical symptoms, Familial Adult-Onset Leukodystrophy manifesting as cerebellar ataxia and dementia, Familial Leukodystrophy with adult onset dementia and abnormal glycolipid storage, Globoid Cell Leukodystrophy (Krabbe Disease), Hereditary Adult Onset Leukodystrophy simulating chronic progressive multiple sclerosis, Hypomyelination with Atrophy of the Basal Ganglia and Cerebellum (HABC), Hypomyelination, Hypogonadotropic, Hypogonadism and Hypodontia (4H Syndrome), Lipomembranous Osteodysplasia with Leukodystrophy (Nasu Disease), Metachromatic Leukodystrophy (MLD), Megalencephalic Leukodystrophy with subcortical Cysts (MLC), Neuroaxonal Leukoencephalopathy with axonal spheroids (Hereditary diffuse leukoencephalopathy with spheroids—HDLS), Neonatal Adrenoleukodystrophy (NALD), Oculodetatoldigital Dysplasia with cerebral white matter abnormalities, Orthochromatic Leukodystrophy with pigmented glia, Ovarioleukodystrophy Syndrome, Pelizaeus Merzbacher Disease (X-linked spastic paraplegia), Refsum Disease, Sjogren-Larssen Syndrome, Sudanophilic Leukodystrophy, Van der Knaap Syndrome (Vacuolating Leukodystrophy with Subcortical Cysts or MLC), Vanishing White Matter Disease (VWM) or Childhood ataxia with diffuse central nervous system hypomyelination, (CACH), X-linked Adrenoleukodystrophy (X-ALD), and Zellweger Spectrum disorders including Zellweger Syndrome, Neonatal Adrenoleukodystrophy, Infantile Refsum Disease, Leukoencephalopathy with brainstem and spinal cord involvement and lactate elevation (LBSL), or DARS2 Leukoencephalopathy. In some embodiments, the leukodystrophy is adrenoleukodystrophy (ALD) (including X-linked ALD), metachromatic leukodystrophy (MLD), Krabbe disease (globoid leukodystrophy), or DARS2 Leukoencephalopathy.

In some embodiments, the subject has an excitotoxicity disorder. Excitotoxicity is a process through which nerve cells become damaged because they are overstimulated. A number of conditions are linked with excitotoxicity including stroke, traumatic brain injury, multiple sclerosis, amyotrophic lateral sclerosis, Alzheimer's disease, and spinal injuries. Damage to the nerve cells results in corresponding neurological symptoms which can vary depending on which cells are damaged and how extensive the damage is. Thus, in some embodiments, the dendrimer/agent complexes are used in imagine or diagnose one or more excitotoxicity disorders.

In some embodiments, the compositions and methods can be used to deliver imaging/diagnostic agents to the site of neuroinflammation, particularly microglial-mediated neuroinflammation. In some embodiments, the disorder is maladaptive neuroinflammation, for example maladaptive inflammation following a traumatic brain injury.

Detecting, Imaging, and/or Treating Cancer In Vivo

Methods of imaging, diagnosing, and/or treating cancer and/or metastatic cancer comprising administering an effective amount of the compositions to a subject in need thereof are also provided. Also disclosed are any of the disclosed dendrimer compositions for use in the localization of cancer and/or metastatic cancer in a subject.

The compositions and methods are useful for imaging, diagnosing, and/or treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

In some embodiments, dendrimers conjugated to one or more radionuclides or one or more MIR contrast agents via ether linkages are used for cancer imaging with Positron Emission Tomography (PET). Exemplary PET imaging agents for cancer imaging include ¹⁸F (Fluorine-18), ⁸⁹Zr (Zirconium-89), ⁹⁰Y (Yttrium-90), and ¹⁷⁷Lu (Luthenium-177). Exemplary MRI imaging agents for cancer imaging includes Gd, Mn, BaSO₄, iron oxides, and iron platinum.

Malignant tumors which may be diagnosed and/or treated are classified according to the embryonic origin of the tissue from which the tumor is derived. Carcinomas are tumors arising from endodermal or ectodermal tissues such as skin or the epithelial lining of internal organs and glands. The compositions are particularly effective in imaging, diagnosing, and/or treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

The types of cancer that can be diagnosed, imaged, and/or treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

Exemplary tumor cells include tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma.

In some embodiments, the subject to be treated is one with one or more solid tumors. A solid tumor is an abnormal mass of tissue that usually does not contain cysts or liquid areas. Solid tumors may be benign (not cancer), or malignant (cancer). Examples of solid tumors are sarcomas, carcinomas, and lymphomas. In some embodiments, the compositions and methods are effective in treating one or more symptoms of cancers of the skin, lung, liver, pancreas, brain, kidney, breast, prostate, colon and rectum, bladder, etc. In further embodiment, the tumor is a focal lymphoma or a follicular lymphoma.

In some embodiments, the subject to be treated is one with a solid tumor of any type or brain metastases. Solid tumors are formed in cancerous tissues in organs within the body. Example organs include, but are not limited to, brain, breast, lung, pancreas, kidney, liver, prostate, ovaries, lungs, thyroid, and pituitary. Brain metastases is a disease in which malignant (cancer) cells originating from another region of the body (e.g. lung or breast) invade the brain and form solid tumor masses.

### Radiopharmaceutical Therapy

Radiopharmaceutical therapy (RPT) is emerging as a safe and effective targeted approach to treating many types of cancer. The compositions and methods are useful for delivering radiation specifically to the targeted cells in the tumor region. In some embodiments, dendrimers conjugated to one or more radionuclides via ether linkages are used for radiopharmaceutical therapy in treating cancer. Suitable radionuclides include both β-particle and α-particle emitters. Exemplary radionuclides for radiopharmaceutical therapy include ¹⁸F (Fluorine-18), ⁸⁹Zr (Zirconium-89), ⁹⁰Y (Yttrium-90), ¹³¹I (iodine-131), ¹⁵³Sm (Samarium-153), ¹⁶⁶Ho, ¹⁷⁷Lu (Luthenium-177), rhenium-186, ²¹¹At, ²¹²Pb, ²²³Ra (Radium-223), ²²⁵Ac, and ²²⁷Th.

Tumor cells take advantage of immunosuppressive mechanisms and establish a strongly immunosuppressive tumor microenvironment (TME), which inhibits antitumor immune responses, supporting the disease progression. Many cell types are thought to contribute to the generation of an immunosuppressive TME including cancer-associated fibroblasts, myeloid-derived suppressor cells (MDSCs), regulatory T cells (Treg), and tumor-associated macrophages (TAMs). In some embodiments, the dendrimer/radionuclides are specifically delivered to one or more reactive immune cells in the tumor region. In some embodiments, the dendrimer/radionuclides are specifically delivered to cancer-associated fibroblasts, MDSCs, Treg, and/or TAMs.

Accordingly, methods of depleting, inhibiting or reducing one or more reactive immune cells at the tumor sites are described. Exemplary reactive immune cells include tumor-permissive and immunosuppressive immune cells, for example, TAMs and MDSCs. Methods of depleting, inhibiting, or reducing tumor associated macrophages (TAMs, or M2-like macrophages) and/or MDSCs at tumor tissues in a subject by selectively delivering radiotherapy to one or more of TAMs and/or MDSCs at tumor tissues are described. The methods include administering to the subject the dendrimer complexes including one or more radionuclides in an effective amount to deplete, inhibit, or reduce quantity of TAMs and/or MDSCs at tumor tissues. In some embodiments, the methods are effective for delivering radiotherapy to tumor site and to kill, deplete, or reduce the target cells as well as surrounding tumor cells.

In some embodiments, the radionuclide labeled dendrimers are further conjugated to one or more anti-tumor drugs. Exemplary anti-tumor drugs include STING agonists, CSF1R inhibitors, PARP inhibitors, VEGFR tyrosine kinase inhibitors, EGFR tyrosine kinase inhibitors, MEK inhibitors, glutaminase inhibitors, TIE II antagonists, and CXCR2 inhibitors.

Exemplary anti-tumor drugs also include Idarubicin, imatinib, irinotecan, exemestane, etoposide, epirubicin, oxaliplatin, octreotide, capecitabine, carboplatin, carmofur, cladribine, clarithromycin, gefitinib, gemcitabine, cyclophosphamide, cisplatin, cytarabine, zinostatin, cetuximab, tamoxifen, daunorubicin, dacarbazine, dactinomycin, tegafur, topotecan, toremifene, doxifluridine, doxorubicin, docetaxel, nimustine, docetaxel, paclitaxel, vincristine, vindensine, vinblastine, nedaplatin, pirarubicin, fluorouracil, flutamide, bleomycin, fadrozole, mitomycin, fludarabine, prednisone, pentostatin, mitoxantrone, medroxyprogesterone, mercaptopurine, mitotane, zinostatin, rapamycin, cyclosporine, mycophenolate mofetil, and mizoribine.

Other exemplary anti-tumor drugs include inhibitors targeting one or more of EGFR, ERBB2, VEGFRs, Kit, PDGFRs, ABL, SRC and mTOR. In some embodiments, one or more anti-tumor drugs are inhibitors such as crizotinib, ceritinib, alectinib, brigatinib, bosutinib, dasatinib, imatinib, nilotinib, vemurafenib, dabrafenib, ibrutinib, palbociclib, sorafenib, ribociclib, cabozantinib, gefitinib, erlotinib, lapatinib, vandetanib, afatinib, osimertinib, ruxolitinib, tofacitinib, trametinib, axitinib, lenvatinib, nintedanib, pazopanib, regorafenib, sunitinib, vandetanib, dacomitinib, and ponatinib. In some embodiments, one or more anti-tumor drugs are tyrosine kinase inhibitors such as HER2 inhibitors, EGFR tyrosine kinase inhibitors. Exemplary EGFR tyrosine kinase inhibitors include gefitinib, erlotinib, afatinib, dacomitinib, and osimertinib.

Further exemplary anti-tumor drugs include anti-angiogenesis agents such as antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (TL-12); thalidomide (THALOMID®) and derivatives thereof such as lenalidomide (REVLIID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

### Controls

The effect of the dendrimer/agent compositions, optionally including one or more additional active agents can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the dendrimer/agent composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same disease or condition as the treated subject.

### Dosages and Effective Amounts

Dosage and dosing regimens are dependent on the severity and location of the disorder or condition and/or methods of administration, and can be determined by those skilled in the art.

In some embodiments, the active agents do not target or otherwise modulate the activity or quantity of healthy cells not within or associated with the diseased/damaged tissue, or do so at a reduced level compared to cells associated with the inflammation or of the tumor region. In this way, by-products and other side effects associated with the compositions are reduced.

A pharmaceutical composition including a therapeutically effective amount of the dendrimer compositions and a pharmaceutically acceptable diluent, carrier or excipient is described. In some embodiments, the pharmaceutical compositions include an effective amount of hydroxyl-terminated PAMAM dendrimers conjugated to one or more radionuclides. The radiotherapy or imaging dose will be determined from clinical studies of subjects with varying degrees of inflammation and/or tumor sizes to determine the optimal dose range.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

The actual effective amounts of dendrimer complex can vary according to factors including the specific active agent administered, the particular composition formulated, the mode of administration, and the age, weight, condition of the subject being treated, as well as the route of administration and the disease or disorder. In some embodiments, the subjects are humans. Generally, the dosage may be lower for intravenous injection or infusion.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly or yearly dosing.

In some embodiments, dosages of dendrimer compositions are administered once, twice, or three times daily, or every other day, two days, three days, four days, five days, or six days to a human. In some embodiments, dosages are administered about once or twice every week, every two weeks, every three weeks, or every four weeks. In some embodiments, dosages are administered about once or twice every month, every two months, every three months, every four months, every five months, or every six months.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### Kits

The compositions can be packaged in kit. The kit can include a single dose or a plurality of doses of a composition including one or more radionuclides encapsulated in, associated with, or conjugated to a dendrimer, and instructions for administering the compositions. Specifically, the instructions direct that an effective amount of the composition be administered to an individual with a particular condition/disease as indicated. The composition can be formulated as described above with reference to a particular treatment method and can be packaged in any convenient manner.

The compositions can be packaged in single or multi-vial kits that contain all of the components needed to prepare the complexes. In some embodiments, a multi-vial kit contains the same general components but employs more than one vial in reconstituting the radiopharmaceutical. It is advantageous that the contents of vials be lyophilized.

The kit also can contain stabilizers, bulking agents such as mannitol, that are designed to aid in the freeze-drying process, and other additives known to those skilled in the art.

The present invention will be further understood by reference to the following non-limiting examples.

### EXAMPLES

### Example 1: Synthesis and Characterization of ¹⁸F Labeled Dendrimer Via Two Synthetic Routes

The ¹⁸F dendrimer (i.e., compound 5, shown in FIG. 1) is synthesized by using either of two chemical routes. For both routes, the first synthetic step is the synthesis of alkyne terminating dendrimer 2 with 10-12 acetylene groups on the surface of dendrimer. As shown in FIG. 1, a generation 4 PAMAM dendrimer is used as an exemplary dendrimer to illustrate the synthetic routes. Briefly, compound 2 is synthesized by partially modifying hydroxyl PAMAM dendrimer (compound 1) by reacting with propargyl bromide in the presence of 2% sodium hydroxide solution in DMSO. The solution was stirred at room temperature for overnight. On reaction completion, the pH was adjusted with saturated ammonium chloride to around 7 and tangential flow filtration (TFF) was performed to purify the product.

The acetylene dendrimer (compound 2) was characterized using ¹H NMR where the internal amides peak between δ 8.05-7.70 ppm is the reference peak. There is a sharp peak corresponding to O—CH₂-acetylene at δ 4.13 ppm and the proton integration method indicated the addition of 10-12 arms of propargyl groups on the dendrimer. HPLC chromatogram showed the shift in retention time from the starting G4-OH dendrimer and the peak for dendrimer (compound 2) was observed at 12.6 minutes. In the next step, 3-azidopropyl 4-methylbenzenesulfonate (compound 3) was clicked to compound (2) using copper catalyzed click chemistry to afford compound (4). The click reaction was performed using CuBr and N,N,N′,N″,N″-pentamethyldiethylenetriamine in the presence of anhydrous DMF by stirring for 2 h. The compound (4) was quickly purified using TFF in water. EDTA wash was also given for the removal of free copper salts. Once again, the reaction completion was monitored through ¹H NMR and HPLC. The ¹H NMR revealed the attachment of 8-10 molecules of compound (3). A multiplet corresponding to aromatic CH from compound (3) at δ 7.49 ppm and a peak corresponding to O—CH₂ triazole at 4.37 ppm, which is formed after the click reaction, were observed. The internal amide protons of dendrimers, triazole protons and Ar protons from the tosyl group appeared in between 68.05-7.70. The HPLC chromatogram of the compound (4) showed a right shift towards the hydrophobic region as compared to compound (3) and appeared at 19.1 minutes (data not shown). Next, the cold fluorine switch of the tosyl group was performed using potassium fluoride, cryptand 222, and anhydrous potassium carbonate in DMSO at 100° C. The reaction was performed for 30 minutes and the product was recovered through quick purification. In the ¹H NMR, the protons corresponding to tosyl group δ7.49 ppm completely disappear and O—CH₂ present next to tosyl group also shifted downfield at δ4.40 ppm which is expected after fluorine switch. The retention time of the cold fluorine dendrimer (5) again shifted back towards the hydrophilic region at 12 minutes in HPLC.

Using the other synthetic route, compound 3 is first labeled with ¹⁸F to obtain fluoro(18)-propyl azide (3a), which is then clicked with dendrimer 2 to achieve final dendrimer compound 5. All the intermediates and final radiolabeled dendrimer are thoroughly characterized.

### Example 2: Plasma Stability Study of Cold Fluorinated Dendrimer

The stability of fluorinated dendrimer in plasma at physiological temperature was evaluated. The fluorinated dendrimer was dissolved in mouse plasma at a concentration of 1 mg/ml and plasma solution was incubated at 37° C. At various time points, 0 hr (control), 2 hr, 5 hr and 8 hr, the fluorinated-dendrimer plasma samples (1 ml) were separated by Amicon Molecular Weight Cut-off filter (3 kDa) by centrifuge. The supernatant was collected and analyzed by Ion Chromatography (IC) for its fluorine content and NH₄F was used as standard. Percentage of fluoride in supernatant at each time point was calculated based on the F- in supernatant and total amount of fluorine of the fluorinated dendrimer.

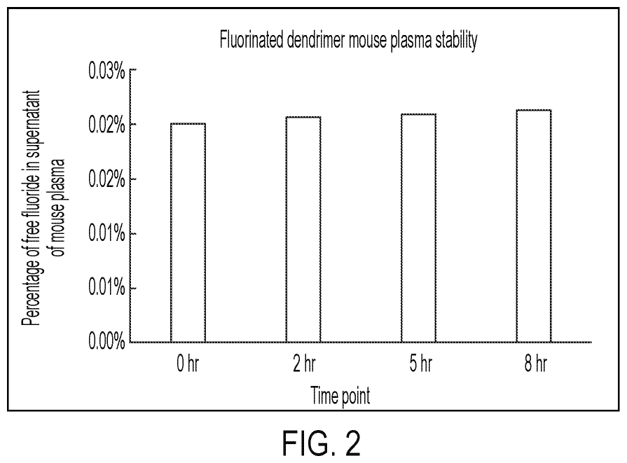

No defluorination (change of fluoride in supernatant) was observed from fluorinated dendrimer after 8 hours of incubation in mouse plasma at 37° C. (FIG. 2), indicating high stably fluorinated dendrimer conjugates.

### Example 3: Synthesis and Characterization of ⁸⁹Zr-Labeled Dendrimer

For the synthesis of the D-deferoxamine-⁸⁹Zr labeled dendrimer 5 (FIG. 3), the synthesis was started from generation 4 hydroxyl-terminated PAMAM dendrimer, PAMAM-G4-OH (1), and etherification was performed using allyl bromide, anhydrous cesium carbonate and tetrabutylammonium iodide in DMF at room temperature. The reaction was stirred at room temperature overnight. On completion, the reaction mixture was purified by performing TFF followed by the lyophilization. ¹H NMR confirmed the formation of compound 2 where 9-10 allyl group arms were attached to the dendrimer. The ¹H NM/IR showed multiplet of —CH corresponding to allyl group at 65.85-5.73 and CH₂ at 65.15 ppm. The purity of the compound (2) was evaluated using HPLC and the peak came at 7.9 minutes with >98% purity. During the next step, a photochemical thiol-ene click was used where cysteamine was added around the allyl group in an anti-Markovnikov fashion. The reaction was performed in anhydrous DMF and 2,2-dimethoxy-2-phenylacetophenone was used as the photoinitiator for the reaction. The reaction was performed under 365 nm UV light for 8 h. On completion, DMF was evaporated and TFF was performed to purify the compound D-amine (3). Once again, the reaction was monitored using ¹H NMR and HPLC. The complete disappearance of allyl peaks in ¹H NMR confirmed the successful completion of the reaction. The ¹H NMR showed —CH₂ peak at 61.6 ppm which was created after the thiol insertion. Once the amine-terminated dendrimer (3) was ready, it was coupled with the p-SCN-Bn-Deferoxamine (DFO) in DMSO. The coupling was performed at pH 7.5 on overnight stirring. The product formation was once again checked by ¹H NMR where the deferoxamine peaks appeared in the NMR after the conjugation. The N-acetyl peak of DFO appeared at 61.97 and —CH₂ peaks from DFO appeared at 61.65-1.15 ppm. The proton integration method was applied to calculate the final loading of the DFO and 9-10 molecules were found to be attached on the dendrimer (compound 4). Once the DFO molecules were attached, HPLC peak corresponding to dendrimer-DFO (4) shifted towards hydrophobic side at 13.1 minutes. The last step is the radiolabeling with hot ⁸⁹Zr with zirconium oxalate where hot Zr molecules is chelated in the DFO arms to afford compound 5. This chelation reaction is performed in HEPES buffer at pH 7.4. ⁸⁹Zr(oxalate)₂ in oxalic acid solution is neutralized with Na₂CO₃ first, then it is incubated with DFO-dendrimer (compound 4). The radiolabeling reaction will be performed for 60 minutes at RT on an agitating heat block. Once the radiolabeling is achieved, quenching of the reaction will be done with 50 mM solution of DTPA. The radiolabeled dendrimer (compound 5) will be purified by HPLC or PD-10 column. The stability of the final construct will be evaluated in mouse and human plasma at physiological conditions.

### Example 4: Chelation of Zirconium to Dendrimer-DFO

Chelation of zirconium in the dendrimer-DFO conjugate was performed and the success of chelation was evaluated using HPLC, NMR and ICP. It was confirmed by all three techniques that the Zr chelation was successfully achieved. HPLC result showed that polarity of Zr-DFO-Dendrimer increases than D4-DFO and the peak is broader after chelation.

The dendrimer-DFO-Zr complex was further analyzed by ¹H NMR which clearly revealed the interaction of DFO protons with Zr by showing the absence of DFO protons due to their limited mobility as a result of chelation.

The Zr chelation was also confirmed via inductively coupled plasma (ICP). Zirconium standard solution was made from a series of dilution of commercial Zr standard (998±3 ppm) by 5% nitric acid (trace metal) solution. The concentration of 0.1 ppm, 0.5 ppm, 1 ppm, 5 ppm, 10 ppm, 20 ppm, 50 ppm and 100 ppm standards solution were prepared for calibration. Zr-DFO-dendrimer (1 mg) was dissolved in 10 ml 5% nitric acid and used for ICP measurement. The ratio of DFO/dendrimer is 10-11/1 while the Zr/dendrimer is 13/1 from ICP measurement.

### Example 5: In Vitro Plasma Stability of Zr-Labeled Dendrimer

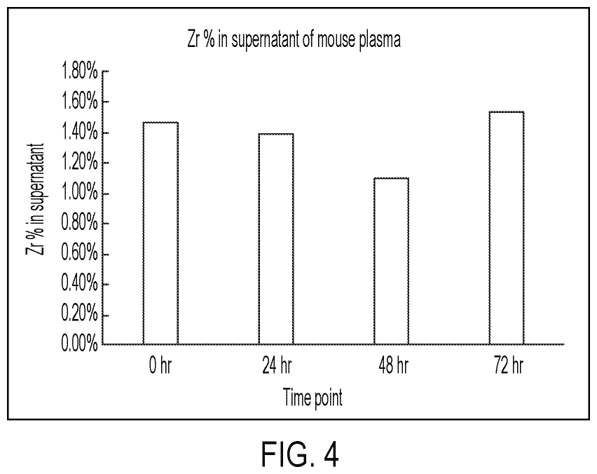

The in vitro plasma stability of Zr-labeled dendrimer at physiological temperature was evaluated. Zr-DFO-dendrimer was dissolved in human or mouse plasma at a concentration of 2 mg/ml and incubated at 37° C. At various time points, e.g., 0 hr (control), 4 hr, 24 hr, 48 hr, and 7 days, the Zr-DFO-dendrimer plasma samples (1 ml) were separated by Amicon Molecular Weight Cut-off filter (3 kDa) by centrifuge. Supernatant which contains free Zr⁴⁺ was collected and diluted (10×) with 5% nitric acid for ICP analysis. The Zr⁴⁺ concentration in plasma supernatant was calculated based on the standard calibration curve by software of ICP instrument. Release percentage of Zr⁴⁺ at each time point was calculated based on the Zr⁴⁺ in supernatant and total amount of Zr in the dendrimer-DFO-Zr conjugate. Less than 2% of Zr release was observed after 72 hours incubation in plasma suggesting highly stable nature of the dendrimer-DFO-Zr conjugate (FIG. 4).

Propargyl arms were introduced on the dendrimer using very mild conditions (2% sodium hydroxide) and the overall procedure is very reproducible and facile. The developed synthetic process is highly adaptable for large-scale synthesis.

### Example 6: Synthesis of ⁹⁰Y Labeled Dendrimer Conjugated to Anti-Tumor Drugs

### Synthesis of ⁹⁰Y Labeled Hydroxyl Dendrimer

For the construction of ⁹⁰yttrium labeled hydroxyl dendrimer, the synthesis began from dendrimer-acetylene (compound 1) which was clicked with Azido-PEG3-amine using copper catalyzed click in the presence of copper sulphate and sodium ascorbate (FIG. 5). The dendrimer (compound 2) was purified using tangential flow filtration and lyophilized. The characterization was done with the help of HPLC and ¹H NMR. Once the amine functionalized dendrimer (compound 2) is ready, it was further reacted with 1,4,7,10-tetraazacyclododecane-1,4,7,10-tetraacetic acid mono-N-hydroxysuccinimide ester (DOTA-NHS) at pH7.5 in DMSO to yield Dendrimer-DOTA (compound 3) where 8-10 arms of DOTA were attached with the dendrimer. In the final step Dendrimer-DOTA was converted to ⁹⁰Y-labeled dendrimer (compound 4) by reacting with hot yttrium in ammonium acetate buffer at pH6.5-7.5. The ⁹⁰Y labeled dendrimer was purified using PD-10 columns or Radio-HPLC.

### Synthesis of 90Y Labeled Dendrimer-Anti-Tumor Drug Combination

It is evident from several preclinical studies that combining immunotherapy and/or chemotherapy with radiotherapy could be a promising strategy for synergistic enhancement of treatment efficacy. Due to the presence of multivalency, dendrimer serve as a potential tool where multiple components (drug, radionuclides, imaging, and ligand) can be introduced precisely on a single dendrimer with high reproducibility and purity. Radiotherapy and anti-tumor drug are to be combined on the same dendrimer construct to have a synergistic effect. Towards that direction, FIG. 6 outlines the synthesis scheme for conjugating both radionuclide and anti-tumor drugs to the dendrimers: first use the 5-6 acetylene arms on the dendrimer (compound 1) to attach the drug and the rest of arms is used to attach DOTA. In the last step, the hot radionuclide is complexed in the DOTA labeled dendrimer-drug conjugate.

### Example 7: ¹⁸F Labeled Dendrimer for Imaging Maladaptive Neuroinflammation in a Mouse Models of Lipopolysaccharide-Induced Sepsis and Neurodegenerative Disease

Myeloid cells, such as reactive macrophages and microglia, and other components of the innate immune response play key roles in the onset and progression of many neurodegenerative diseases. While PET imaging provides a non-invasive method to visualize and quantify inflammation in the brain and periphery (Jain, P. et al., JNM. 2020, 62(8)1107-1112), the current gold-standard PET tracer, TSPO, does not specifically image macrophage activity.

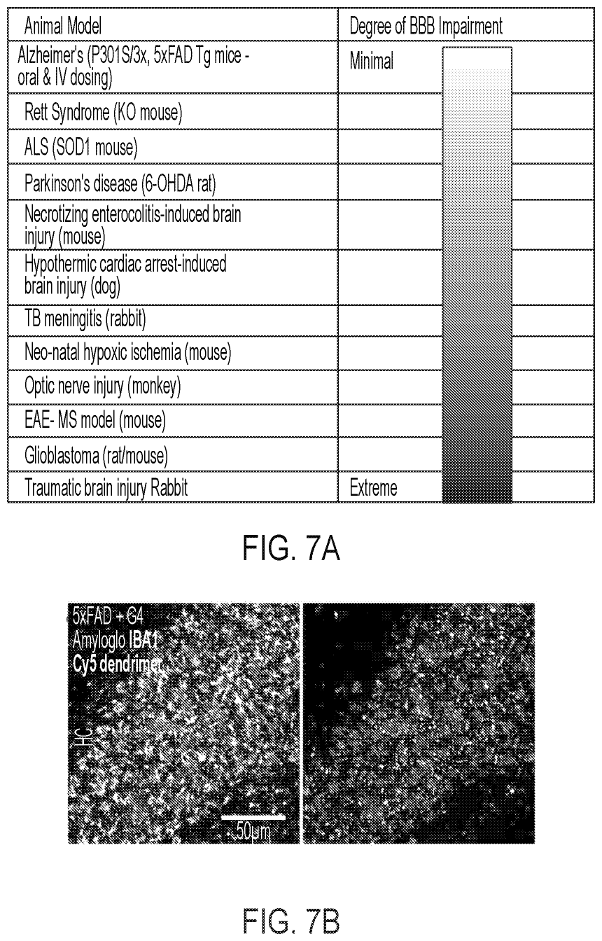

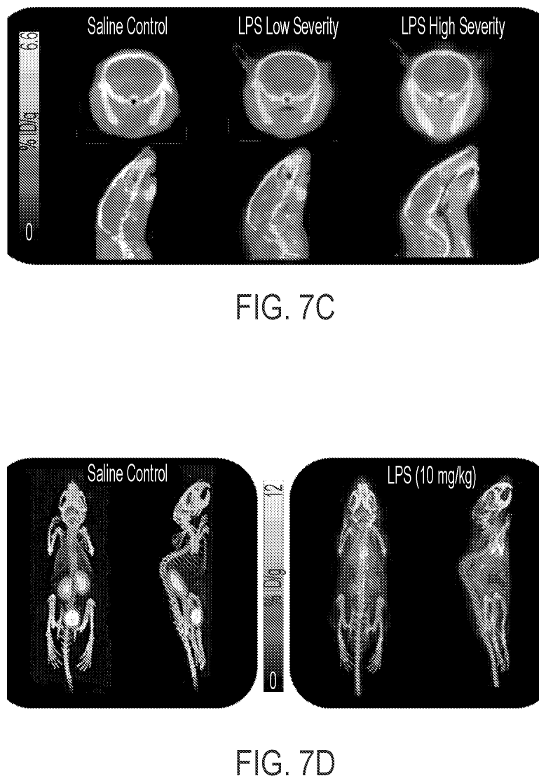

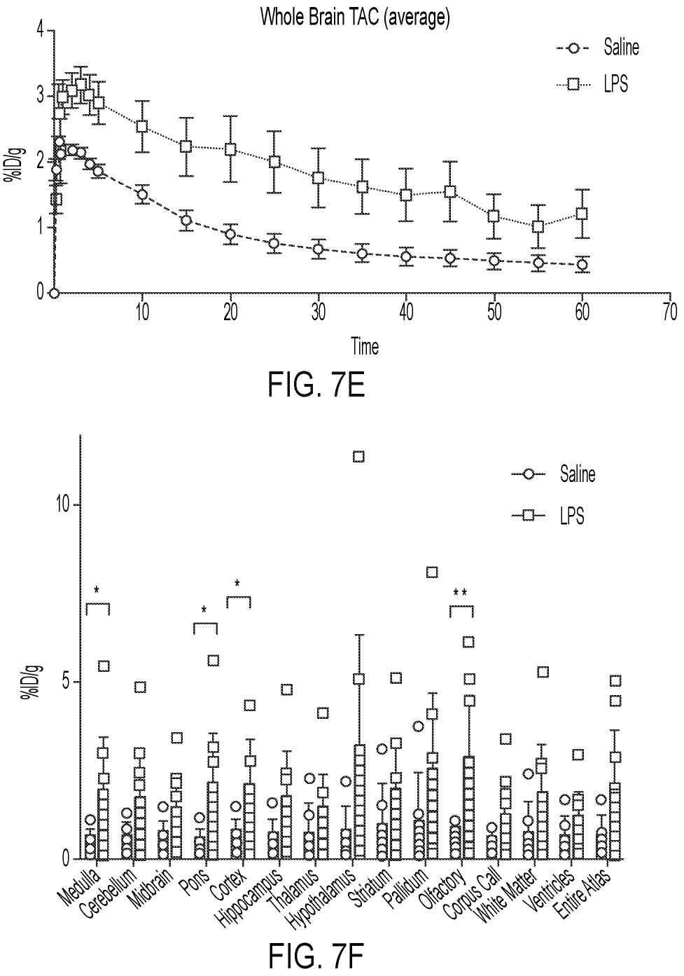

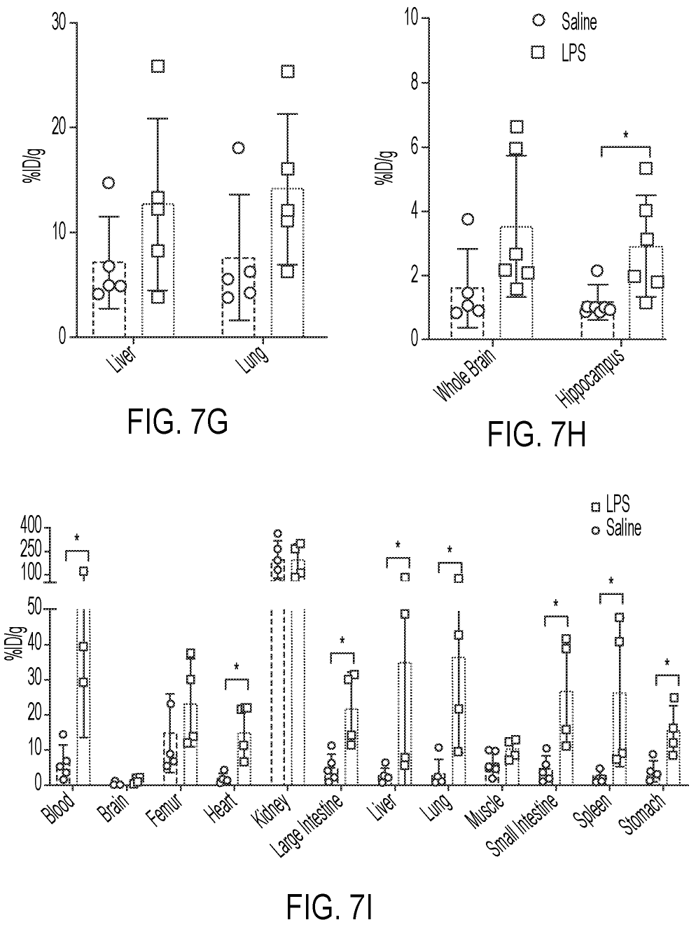

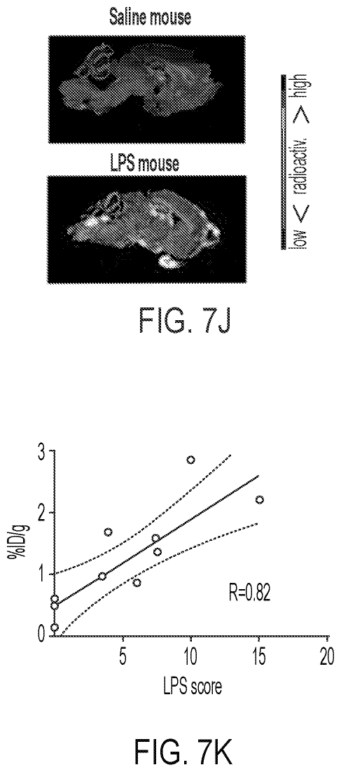

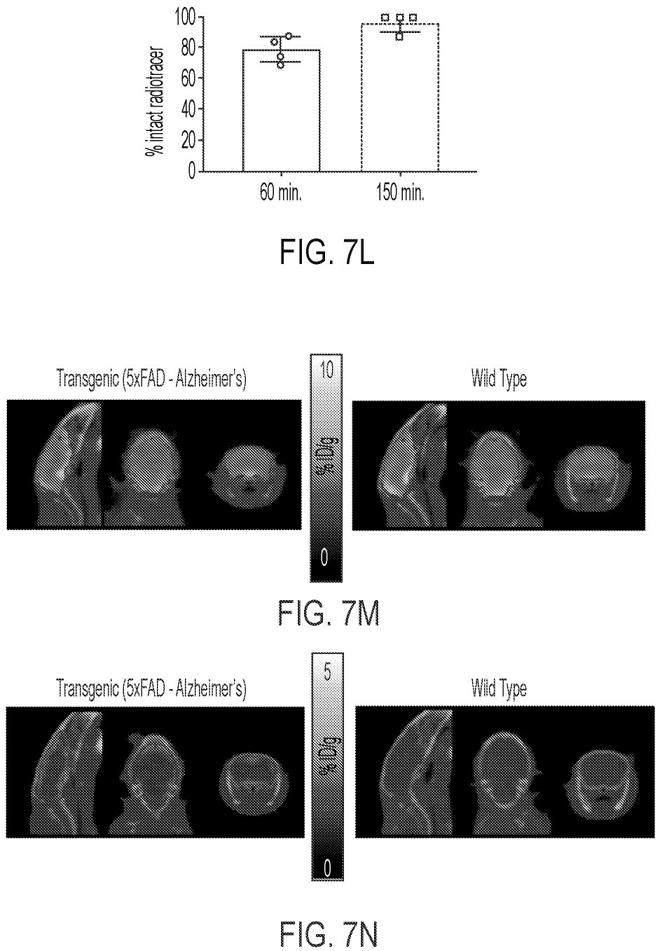

OP-801, a synthetic PAMAM hydroxyl dendrimer, crosses the BBB in the presence of inflammation and is selectively taken up (>95%) by reactive microglia and macrophages. After systemic administration, these hydroxyl dendrimers (without a need for any ligands or antibodies) cross the blood brain barrier in the presence of neuroinflammation and selectively target reactive microglia/macrophages and astrocytes, with minimal uptake in healthy brains and tissues. Fluorescently labeled dendrimers are successfully measured in reactive microglia in animals with neuroinflammation. These hydroxyl dendrimers are selectively endocytosed by reactive microglia in test animals demonstrated by >95% of reactive microglia containing dendrimers within 4 hours after a systemic dose administration. As shown in FIG. 7A, the extent of the uptake of hydroxyl dendrimer in reactive microglia correlates with the degree of neuroinflammation and has been quantitatively assessed in models of acute neuroinflammation of varying severity. These nonclinical studies include testing the ALS mouse model with the superoxide dismutase 1 gene mutation (ALS (SOD1 mouse)).

The current study aims to develop the first hydroxyl dendrimer-based PET tracer targeting reactive microglia and evaluates its potential for detecting subtle inflammation in a mouse model of lipopolysaccharide (LPS)-induced sepsis.

### Methods

In the 5×FAD transgenic mouse model, 7-month-old mice were administered IV with G4 hydroxyl dendrimers conjugated with a Cy5 label. Mice were sacrificed at 48 hours post-dose and perfused with saline. Histology of the brain indicated selective uptake of the hydroxyl dendrimer by reactive microglia around the beta amyloid plaque with no detectable uptake by oligodendrocytes or neurons and, there are no detectable uptake by normal microglia as demonstrated in FIG. 7B. The degree of selectivity for reactive microglia over normal microglia suggest that OP-801 will be more selective for regions of neuroinflammation than current imaging approaches that are based upon protein expression such as translocator protein (TSPO) present in both normal and reactive microglia and astrocytes.

To translate the histological findings into an in vivo molecular imaging approach, hydroxyl dendrimer was radiolabeled via 2-step azide fluorination and Cu-catalyzed click reaction as shown in FIG. 1 to provide ¹⁸F Labeled hydroxyl dendrimer (compound 10 of FIG. 1, also referred to as ¹⁸F-OP-801). The product was formulated for injection using SEC, and 150-250 μCi (5-10 ng of ¹⁸F-OP-801) was injected per mouse 24 hours after intraperitoneal injection administration of 10 mg/kg of LPS (n=6) or saline (n=5). Plasma stability was evaluated 60 minutes (n=4) and 150 minutes (n=4) post-injection. Dynamic 60-minute PET images were acquired, and static images were taken by binning 25-35 or 50-60 minute counts. Two LPS mice with very low murine sepsis scores (Shrum, B. et al., BMC Res Notes. 2014, 7(233)) were excluded from the analysis. After imaging, mice were perfused, and organs dissected; radioactivity was measured using a gamma counter. Tracer distribution within the brain was evaluated using autoradiography and Nissl staining of sagittal brain slices. Adult female mice were used for all studies.

### Results

As shown in FIGS. 7C and 7D, normal (saline) mice rapidly cleared ¹⁸F-OP-801 through the kidneys into the bladder consistent with results of another D4 hydroxyl dendrimer administered to human healthy volunteers (data not shown). In contrast, LPS treated mice had significant uptake of ¹⁸F-OP-801 throughout the peritoneal cavity and in the brain correlating with the degree of inflammation. LPS causes activation of macrophages and microglia which are the target of ¹⁸F-OP-801 and hydroxyl dendrimers in general.

Static 50-60 minute summed PET images revealed linearly increasing ¹⁸F-OP-801 uptake with composite MSS score (R²=0.94). Dynamic image time-activity curves showed that the optimal static imaging time is between 25 and 60 minutes post injection of ¹⁸F-OP-801 (FIG. 7E). Brain atlas analysis of 50-60 min summed PET images showed that uptake differed significantly (p<0.05) between LPS and saline treated mice in the cortex, medulla, olfactory bulb, and pons (FIG. 7F). Hippocampal uptake differed significantly (p=0.045) between LPS- and saline-treated mice, as demonstrated by 25-35 min static images (FIGS. 7G and 7H). Ex vivo biodistribution data and PET showed similar uptake patterns for LPS (n=4) vs. saline (n=5) mice (FIG. 7I), as described in further detail below.

To determine potential dose limiting organs (i.e., radiation dosimetry), imaging-based assessment and ex vivo gamma counting of ¹⁸F-OP-801 biodistribution in mice was performed. In brief, ¹⁸F-OP-801 (150-250 Ci) was administered intravenously to healthy female C57BL/6 mice (n=30). Four mice were euthanized at each timepoint: 5, 15, 30, 60, 90, 270 minutes without perfusion. Organs were dissected, weighed, and placed into a gamma counter to measure radioactivity. For image-based dosimetry studies, dynamic 60 minute PET/CT imaging was conducted in healthy male (n=5) and healthy female (n=3) C57BL/6 mice, followed by static 5-minute scans at 90 minutes, and 270 minutes. Regions of interest (ROIs) were manually drawn over organ in each image to quantify radioactivity within each structure. These data (image-based quantification for male organs and image-plus gamma counter-based quantification for females) were converted to estimated human % ID/g and entered into OLINDA. Based on preliminary PET imaging of healthy control female mice, the dose-limiting organs are expected to be the kidneys and bladder (FIG. 7I). These results were corroborated by the gamma counting biodistribution study. Significant differences were observed in organs expected to have an increased immune response, including liver (2.7±2.20 saline vs. 35.2±35.14 LPS % ID/g, p=0.032), lung (2.9±4.34 vs. 36.4±27.27% ID/g, p=0.032), and spleen (2.0±1.60 vs. 26.2±20.98% ID/g, p=0.016).

Autoradiography results corroborated brain uptake from PET images (FIG. 7J). A plot of LPS MSS scores versus % ID/g in whole brain from 50-60 minute PET, with linear regression and 95% confidence intervals is shown in FIG. 7K. These results indicate the ability to selectively image systemic inflammation as well as neuroinflammation using hydroxyl dendrimers such as ¹⁸F-OP-801.

Plasma stability and dosimetry was also conducted in mice. Ex vivo plasma stability of ¹⁸F-OP-801 in healthy mice was evaluated at 60- and 150-minutes post-injection (n=4/group). Plasma stability was >95%, on average, in female mice after 150 minutes (FIG. 7L).

In summary, the current study shows that labeled hydroxyl dendrimers are highly specific PET tracer with extraordinary potential for non-invasive molecular imaging of maladaptive inflammation, including imaging of ALS and Alzheimer's patients.

To further evaluate the ability of ¹⁸F-OP-801 to image neuroinflammation in neurogenerative diseases, the 5×FAD transgenic mouse model was used. 5×FAD transgenic mice (B6SJL-Tg (APPSwFlLon,PSEN1*M146L*L286V)6799Vas) expressed human APP and PSEN1 transgenes developing amyloid plaque within a few months after birth and previous studies with TSPO PET radiotracers have only been able to detect neuroinflammation around 6 months (˜26 weeks) of age with a high TPSO affinity radiotracer, ¹⁸F-GE180. 5×FAD female mice and age matched controls were used at 3.75 months of age to evaluate ¹⁸F-OP-801 compared to the TPSO radiotracer, ¹⁸F-GE180. ¹⁸F-OP-801 (150-250 Ci) was injected as an IV bolus into wild type (n=7) and 5×FAD females (n=12) at 3.75 months of age. ¹⁸F-GE180 (150-250 μCi) was administered to the 5×FAD transgenic (n=5) and wild type (n=4) female mice at 3.75 months of age. Static 10-minute PET/CT images were acquired at 50-60 minutes post-injection. VivoQuant brain atlas was overlaid on CT images and registered to PET to quantify uptake in specific brain regions. ¹⁸F-OP-801 yielded a 3-fold higher PET signal in the 5×FAD mice compared to the wild type mice whereas ¹⁸F-GE180 provided minimal PET signal differences between the 5×FAD mice and wild type controls (FIGS. 7M and 7N; Table 1).

TABLE 1
Comparison between transgenic (TG)-to-wild type (WT) ratios
(equivalent to signal-to-background ratios) in brain regions
known to present amyloid pathology in 5xFAD mice treated with either
¹⁸F-OP-801 or ¹⁸F-GE180 at 3.75 months of age.

| | ¹⁸F-OP-801 | ¹⁸F-GE180 |
|---|---|---|
| | (n = 4 WT, | (n = 4 WT, |
| | n = 4 TG) | n = 5 TG) |
| Cortex | 3.18 | 1.21 |
| Hippocampus | 3.05 | 1.24 |
| Whole Brain | 3.14 | 1.21 |

When ¹⁸F-OP-801 was administered to mice at 5 months of age, a 4-fold higher PET signal was observed in 5×FAD mice compared to wild type controls (FIG. 7O; Table 2). At 5 months, significant differences were observed between the 5×FAD transgenic (TG) mice and wild type (WT) mice in ¹⁸F-OP-801 uptake in cortex (p=0.005) (TG: 0.26±0.095 SUV, WT: 0.11±0.041 SUV), hippocampus (p=0.017) (TG: 0.18±0.065 SUV, WT: 0.10±0.026 SUV) and whole brain (p=0.004) (TG: 0.20±0.082 SUV, WT: 0.10±0.039 SUV). Taken together, these results demonstrate that ¹⁸F-OP-801 can detect early stage neuroinflammation with high sensitivity, due to its excellent selectivity compared to the lower selectivity of TSPO PET radiotracers and provides information on the progression of neuroinflammation over time.

TABLE 2
Transgenic (TG)-to-wild type (WT) ratios (equivalent to signal-to-
background ratios) in brain regions known to present amyloid pathology in
5xFAD mice treated with ¹⁸F-OP-801 at 5 months of age.

| | ¹⁸F-OP-801 |
|---|---|
| | (n = 4 WT, |
| | n = 4 TG) |
| Cortex | 4.75 |
| Hippocampus | 4.64 |
| Whole Brain | 4.74 |

A GLP toxicology study in Sprague Dawley rats was conducted with OP-801 (cold) at more than a 1000-fold safety factor to the anticipated human mass dose to be administered as a single dose (Day 1) to replicate the projected human dose exposure from the imaging treatment (Table 3). The GLP study was conducted at Charles River Laboratories with 10 male and 10 female Sprague Dawley rats per group. Test groups included vehicle control, low, medium and high dose of OP-801 (cold) administered on Day 1. Five (5) rats per sex per group were euthanized on Days 2 and 13. Clinical pathology, gross necropsy, and histology on full sets of standard tissues and gross lesions were conducted on each test animal after sacrifice. Additional rats (6 per sex per group) were evaluated for toxicokinetics. Toxicokinetic analysis was conducted by AIT Biosciences using a validated LC/MS method to detect OP-801 in rat plasma.

TABLE 3
Single dose rat GLP toxicology study design for OP-801 (Study 3101-011)

| | | Dose | | | | |
|---|---|---|---|---|---|---|
| | | Level | Dose | Dose | Main Study | |
| Group | Test | (mg/kg/ | Volume^{a} | Concentration | No. of | No. of |
| No. | Material | day) | (mL/kg) | (mg/mL) | Males | Females |
| 1 | Vehicle | 0 | 1 | 0 | 10 | 10 |
| | Control^{b} | | | | | |
| 2 | OP-801^{b} | 0.2 | 1 | 0.2 | 10 | 10 |
| 3 | OP-801^{b} | 1 | 1 | 1 | 10 | 10 |
| 4 | OP-801^{c} | 5 | 1 | 5 | 10 | 10 |

No. = Number
^{a}Based on the most recent practical body weight measurement.
^{b}5/sex/group will be necropsied on Day 2 and 8.
^{c}5/sex/group will be necropsied on Day 2 and 13.

| | | Dose | | | | |
|---|---|---|---|---|---|---|
| | | Level | Dose | Dose | Toxicokinetic Study | |
| Group | Test | (mg/kg/ | Volume^{a} | Concentration | No. of | No. of |
| No. | Material | day) | (mL/kg) | (mg/mL) | Males | Females |
| 5 | Vehicle | 0 | 1 | 0 | 3 | 3 |
| | Control | | | | | |
| 6 | OP-801 | 0.2 | 1 | 0.2 | 6 | 6 |
| 7 | OP-801 | 1 | 1 | 1 | 6 | 6 |
| 8 | OP-801 | 5 | 1 | 5 | 6 | 6 |

No. = Number
^{a}Based on the most recent practical body weight measurement.

### Endpoints—GLP Rat Study

Mortality checks (daily, am and pm).
Clinical Observations (1 and 4 hours postdose) and daily on non-dosing days.
Body weights (Daily).
Food Consumption (Daily).
Ophthamologic examinations (pretreatment, prior to Day 15 termination).
Necropsy and recording of macroscopic findings (Days 2, 9, and 15).
Organ weights.
Histopathology.
   Comprehensive list of tissues from all control and high-dose animals as well as gross lesions and target organs from lower dose groups.
   Toxicokinetics (following dosing on Days 1 and 8).

### Example 8: Hydroxyl Dendrimer-Based SPECT Imaging Agent, ¹¹¹In-D6-B483, for the Targeted Imaging of Tumors in an Orthotopic Mouse Glioblastoma Multiforme Model

Hydroxyl dendrimers (HDs) are taken up by tumor associated macrophages and microglia (TAMs) with selectivity superior to antibodies in animal models of orthotopic glioblastoma multiforme (GBM). The initial efforts to develop ¹¹¹In-D6-B483, a HD with covalently linked DOTA attached to the surface hydroxyl groups, indicated the potential for targeting orthotopic GBM in a mouse model but required further optimization for clinical use (Liaw et al, Bioeng Transl Med 2020; 5(2): 1-12 (https://doi.org/10.1002/btm2.10160); Cleland et al, Cancer Res 2020; 80(16 Suppl): Abstract 679; Cleland et al, Clin Cancer Res 2021; 27(8Suppl): Abstract PO-097)

Current approaches for imaging of GBM rely on magnetic resonance imaging (MRI) to diagnose and grade the glioma status. ¹¹¹In-D6-B483 has the potential to quantify the degree of TAM involvement that may correlate to the severity of the glioma. In addition, D6-B483 may be used for radiotherapy using ⁹⁰Y in place of ¹¹¹In. HDs have been observed to be retained in TAMs for up to one month after a single administration providing a local reservoir of radiation with systemic exposure (systemic clearance within 2 days).

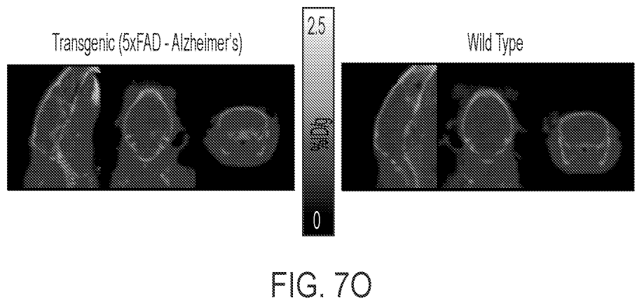

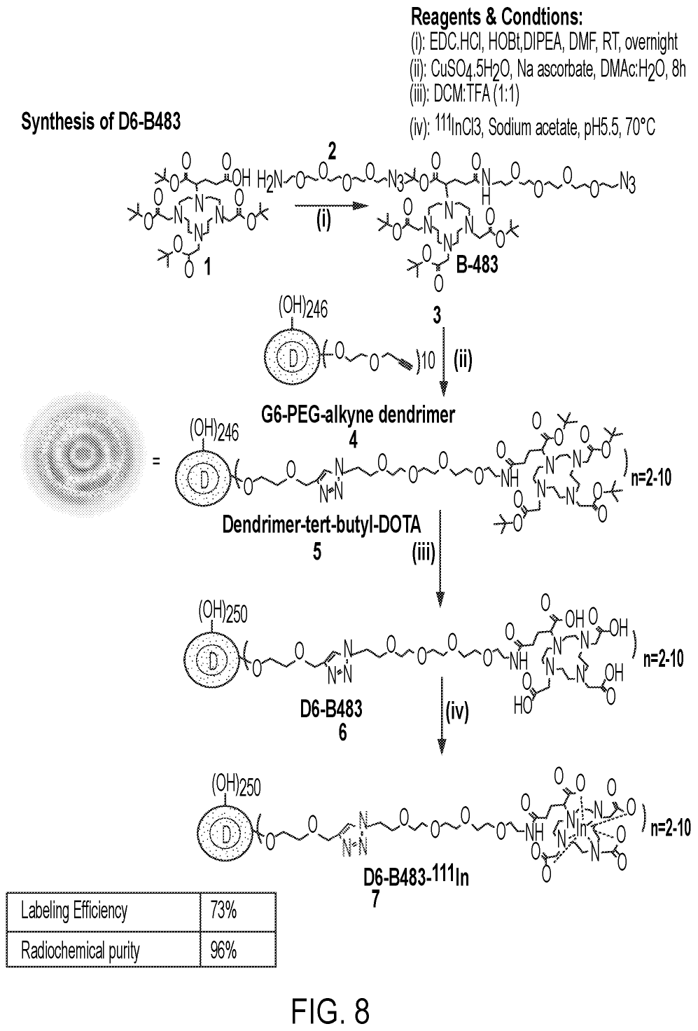

Methods: The synthesis of D6-B483 was achieved in two reaction steps. During the first synthetic step, partial propargylation of the dendrimer was achieved using sodium hydride and propargyl bromide. In the second step, azide-terminated DOTA was attached to propargyl dendrimer to yield D-B483. FIG. 8 shows an example of a scheme and reaction conditions used in a synthesis of ¹¹¹In-D6-B483. For the synthesis of Cy5 labeled dendrimer, 1-2 propargyl functional groups on dendrimer were reacted with Cy5 azide to achieve Cy5-D6-B483. Cy5 labelled D6-B483 with either 2-3 or 8-10 DOTA (10 mg/kg) was administered IV to mice. Mice (3/timepoint) were sacrificed at 15 min, 4, 24, 48, and 96 h post-dose. Amount of Cy5-D6-B483 was measured in kidney and liver after tissue homogenization and extraction (Liao 2020). D6-B483 was mixed with ¹¹¹InCl₃ heated to 85° C. to yield a labeling efficiency of 73%. Using centrifuge membrane system (3 kDa cut off), the product was buffer exchanged to remove free ¹¹¹In and formulated for injection in PBS. The final product had a radiochemical purity of 96%.

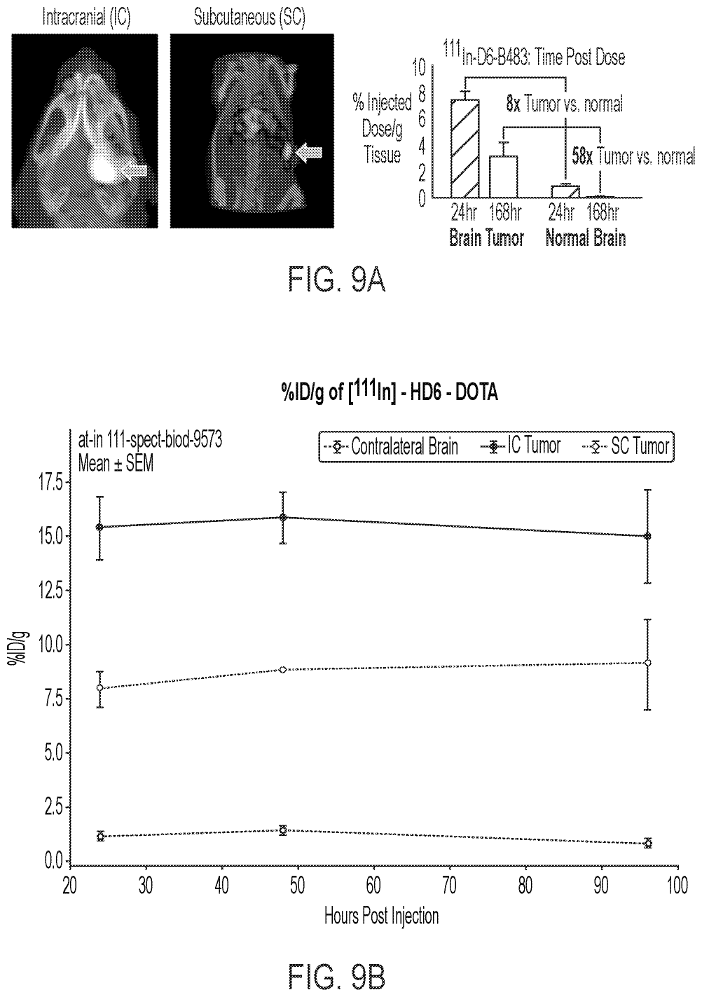

The selective uptake of ¹¹¹In-D6-B483 was evaluated in brain and solid tumors. Twenty female mice were implanted with 10⁶ GL-261-luc2 cells by stereotactic intracranial (IC) surgery. Brain tumor size and location was measured by bioluminescence (BLI) to confirm tumors were between 15 to 60 mm³ prior to dosing. A separate group of 8 mice were implanted subcutaneous (SC) with 10⁶ GL-261-luc2 cells and dosed once tumors were between 125 to 350 mm³ (caliper measurements). All mice received a single IV dose of ¹¹¹In-D6-B483 (˜230 μCi, 45 μg) and SPECT/CT images were obtained at 3-6, 24, 48, 72 and 96 h post-dose. Example results are shown in FIG. 9A. Within 24 h after terminal sacrifice, gamma counting of tissues was performed.

Results: HDs have previously been demonstrated in animals and humans to be renally secreted with minimal liver uptake (Lesniak et al, Mol. Pharmaceutics 1013; 10: 4560-4571). D6-B483 with 2-3 DOTA had less exposure to the liver than D6-B483 with 8-10 DOTA with comparable kidney exposure. Stability of the ¹¹¹In-D6-B483 in mouse serum out to 4 days was greater than 90% parent. Bioluminescence imaging was used to enroll 12 IC and 4 SC tumored mice on study. SPECT/CT imaging showed relatively high uptake of ¹¹¹In-D6-B483 in the IC and SC tumors (approximately 15% ID/g and 8% ID/g respectively, as shown in FIG. 9B) compared with approximately 1% ID/g in contralateral brain.

Conclusion: ¹¹¹In-D6-B483 is a targeted and highly selective SPECT tracer for non-invasive imaging of brain tumors enabling precise quantitation of HD uptake and corresponding TAM involvement. ¹¹¹In-D6-B483 is currently being developed for a Phase 1 study in GBM and brain metastases patients.

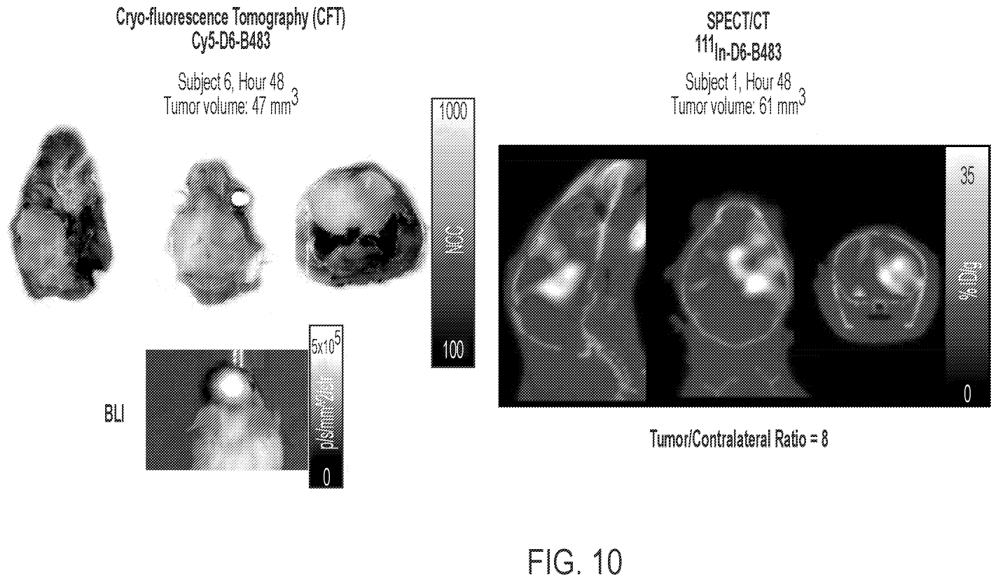

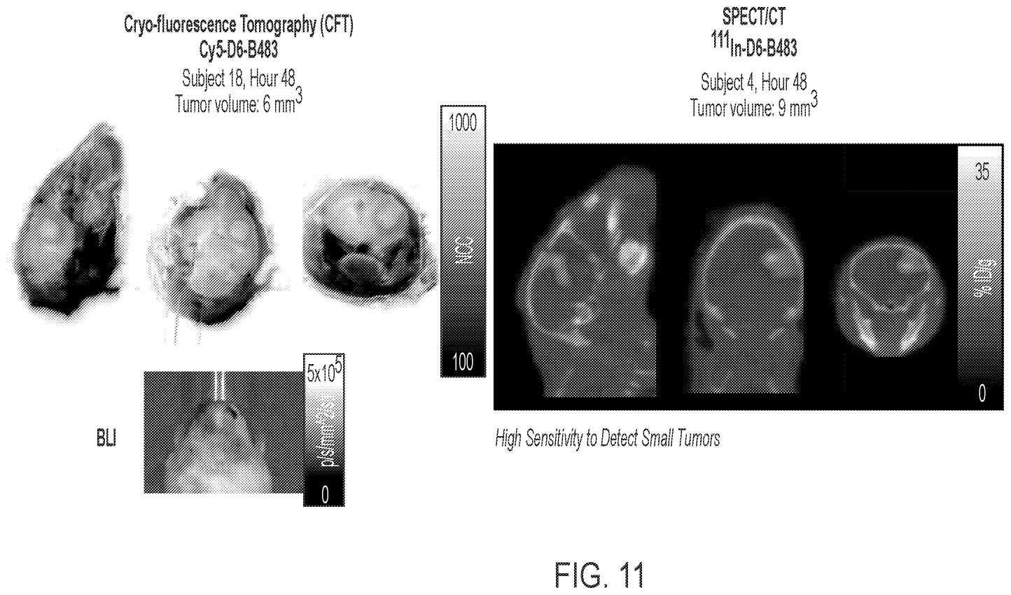

The localization of ¹¹¹In-D6-B483 in large tumors (FIG. 10) and small tumors (FIG. 11) was also evaluated. Large tumors were imaged by cryo-fluorescence Tomography (CFT), which showed localization of Cy5-D6-B483 after 48 h (tumor size: 47 mm) (FIG. 10, left panel). Using ¹¹¹In-D6-B483, a Tumor/Contralateral Ratio=8:1 was found when imaging large tumor after 48 h (tumor size: 61 mm) (FIG. 10, right panel). Small tumors were also imaged using Cryo-fluorescence Tomography (CFT), and localization of Cy5-D6-B483 was observed at time=48 h (tumor size: 6 mm) (FIG. 11, left panel). ¹¹¹In-D6-B483 was also used to image the tumor after 48 h (tumor size: 9 mm), showing a high sensitivity to detect small tumors (FIG. 11, right panel).

Those skilled in the art will recognize, or be able to ascertain using no more than routine experimentation, many equivalents to the specific embodiments of the invention described herein. Such equivalents are intended to be encompassed by the following claims.

We claim:

1. A composition comprising hydroxyl-terminated dendrimers conjugated to one or more radionuclides or one or more magnetic resonance imaging (MRI) contrast agents through ether linkages, optionally via one or more linking moieties.

2. The composition of claim 1, wherein the dendrimers are polyamidoamine (PAMAM) dendrimers, polypropylamine (POPAM) dendrimers, polyethylenimine dendrimers, polylysine dendrimers, polyester dendrimers, iptycene dendrimers, aliphatic poly(ether) dendrimers, or aromatic polyether dendrimers.

3. The composition of claim 2, wherein the dendrimers are generation 4, generation 5, generation 6, generation 7, or generation 8 PAMAM dendrimers.

4. The composition of any one of claims 1-3, wherein the one or more radionuclides are selected from the group consisting of ¹⁸F, ⁵¹Mn, ⁵²Fe, ⁶⁰Cu, ⁶⁸Ga ⁷²As, ^{94m}Tc, or ¹¹⁰In, ¹⁸F, ¹²⁴I, ¹²⁵I, ¹³¹I, ¹²³I, ⁷⁷Br, ⁷⁶Br, ^{99m}Tc, ⁵¹Cr, ⁶⁷Ga ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ²²⁵Ac, ¹⁹⁸Au, and ¹⁹⁹Au, or ⁸⁹Zr.

5. The composition of any one of claims 1-4, wherein the one or more radionuclides are ¹⁸F, ⁸⁹Zr, ⁹⁰Y, or ¹⁷⁷Lu.

6. The composition of any one of claims 1-3, wherein the one or more MRI contrast agents are selected from a group consisting of Gd, Mn, BaSO₄, iron oxides, and iron platinum.

7. The composition of any one of claims 1-3 and 6, wherein the MRI contrast agent is Gd.

8. The composition of any one of claims 1-7 in an amount effective to selectively accumulate within reactive microglia and/or macrophages at sites of inflammation or in reactive immune cells in tumors.

9. The composition of any one of claims 1-7, further comprising one or more additional active agents.

10. A composition comprising a compound that comprises a dendrimer conjugated to a radionuclide or an MRI contrast agent through an ester, ether, or amide linkage, wherein the dendrimer comprises a high density of surface hydroxyl groups.

11. The composition of claim 10, wherein the radionuclide or the MRI contrast agent is conjugated to the ester, ether, or amide linkage through a spacer.

12. The composition of claim 11, wherein the spacer comprises alkyl groups, heteroalkyl groups, or alkylaryl groups.

13. The composition of claim 11 or 12, wherein the spacer comprises a peptide.

14. The composition of any one of claims 11-13, wherein the spacer comprises polyethylene glycol.

15. The composition of any one of claims 10-14, wherein conjugation of the radionuclide or the MRI contrast agent occurs on less than 50% of total available surface functional groups of the dendrimer prior to the conjugation.

16. The composition of any one of claims 10-15, wherein conjugation of the radionuclide or the MRI contrast agent occurs on less than 5%, less than 10%, less than 20%, less than 30%, or less than 40% of total available surface functional groups of the dendrimer prior to the conjugation.

17. The composition of any one of claims 10-16, wherein the radionuclide is selected from the group consisting of ¹⁸F, ⁵¹Mn, ¹²Fe, ⁶⁰Cu, ⁶⁸Ga, ⁷²As, ^{94m}Tc, ¹¹⁰In, ¹⁸F, ¹²⁴, ¹²⁵, ¹³¹, ¹²³, ⁷⁷Br, ⁷⁶Br, ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ²²⁵Ac, ¹⁹⁸Au, ¹⁹⁹Au, and ⁸⁹Zr.

18. The composition of claim 17, wherein the radionuclide is ¹⁸F, ⁸⁹Zr, ⁹⁰Y, or ¹⁷⁷Lu.

19. The composition of any one of claims 10-16, wherein the MRI contrast agent is selected from the group consisting of Gd, Mn, BaSO₄, iron oxides, and iron platinum.

20. The composition of claim 19, wherein the MRI contrast agent is Gd.

21. The composition of any one of claims 10-20, wherein the dendrimer is selected from the group consisting of polyamidoamine (PAMAM) dendrimers, polypropylamine (POPAM) dendrimers, polyethylenimine dendrimers, polylysine dendrimers, polyester dendrimers, iptycene dendrimers, aliphatic poly(ether) dendrimers, and aromatic polyether dendrimers.

22. The composition of any one of claims 10-21, wherein the zeta potential of the compound is between −25 mV and 25 mV.

23. The composition of any one of claims 10-22, wherein the zeta potential of the compound is between −20 mV and 20 mV, between −10 mV and 10 mV, between −10 mV and 5 mV, between −5 mV and 5 mV, or between −2 mV and 2 mV.

24. The composition of any one of claims 10-23, wherein the surface charge of the compound is neutral or near-neutral.

25. The composition of any one of claims 10-24, wherein the dendrimer is conjugated to the radionuclide or the MRI contrast agent through an ether or amide linkage.

26. The composition of any one of claims 10-25, wherein the dendrimer is conjugated to the radionuclide or the MRI contrast agent through an ether linkage.

27. A pharmaceutical composition comprising the composition of any one of claims 1-26 and a pharmaceutically acceptable excipient.

28. The pharmaceutical composition of claim 27, formulated for intraperitoneal, intravenous, intrathecal, intratumoral, or oral administration.

29. A method for detecting or imaging one or more inflammatory and/or cancer cells in a subject in need thereof, the method comprising administering to the subject an effective amount of a composition comprising a compound that comprises a dendrimer conjugated to a radionuclide or an MRI contrast agent through an ester, ether, or amide linkage, wherein the dendrimer comprises a high density of surface hydroxyl groups.

30. The method of claim 29, wherein the composition is administered systemically to the subject.

31. The method of claim 29 or claim 30, wherein the composition is administered via intraperitoneal, intravenous, intrathecal, intratumoral, or oral route.

32. The method of any one of claims 29-31, wherein the composition is administered in an amount effective to detect, diagnose or monitor one or more inflammatory sites or cancer in the subject.

33. The method of any one of claims 29-32, wherein the one or more inflammatory sites in the subject are associated with one or more inflammatory diseases, or associated with sepsis or septic shock, or caused by any mechanism of macrophage activation including macrophage activation syndrome.

34. The method of any one of claims 29-32, wherein the one or more inflammatory sites in the subject are associated with one or more autoimmune diseases or disorders.

35. The method of claim 34, wherein the one or more autoimmune diseases or disorders are selected from the group consisting of rheumatoid arthritis, psoriasis, psoriatic arthritis, multiple sclerosis, Sjögren syndrome, Myasthenia gravis, Graves disease, Addison disease, Celiac disease, Hashimoto thyroiditis, Pernicious anemia, Guillain-Barre syndrome, Chronic inflammatory demyelinating polyneuropathy, vasculitis, systemic lupus erythematosus (SLE), type 1 diabetes, inflammatory bowel disease, and thyroid diseases.

36. The method of any one of claims 29-32, wherein the one or more inflammatory sites in the subject include one or more sites of neuroinflammation in the central nervous system.

37. The method of claim 36, wherein the one or more sites of neuroinflammation in the central nervous system in the subject are associated with Alzheimer's disease.

38. The method of any one of claims 29-32, wherein the one or more inflammatory sites in the subject are associated with amyotrophic lateral sclerosis (ALS).

39. The method of any one of claims 29-32, wherein the subject has cancer.

40. The method of claim 39, wherein the cancer is solid tumor of any type or brain metastases.

41. The method of any one of claims 29-40, wherein the method further comprises the step of imaging the subject with a molecular imaging device to detect the dendrimers conjugated to one or more radionuclides in the subject, wherein detection of the dendrimers conjugated to one or more radionuclides indicates presence of inflammation or cancer cells.

42. The method of claim 41, wherein the molecular imaging device comprises a gamma camera for positron emission tomography (PET) scanning or a scanner for magnetic resonance imaging.

43. A method for detecting or imaging one or more inflammatory and/or cancer cells in a subject in need thereof, the method comprising administering to the subject an effective amount of the composition of any one of claims 1-28.

44. A method for treating cancer comprising administering to a subject in need thereof an effective amount of the composition of any one of claims 1-28.

45. A method for treating cancer comprising administering to a subject in need thereof an effective amount of a composition comprising a compound that comprises a dendrimer conjugated to a radionuclide or an MRI contrast agent through an ester, ether, or amide linkage, wherein the dendrimer comprises a high density of surface hydroxyl groups.

46. The method of claim 45, wherein the wherein the composition is administered systemically to the subject.

47. The method of claim 45 or claim 46, wherein the composition is administered via intraperitoneal, intravenous, intrathecal, intratumoral or oral route.

48. The method of any one of claims 45-47, wherein the cancer is breast cancer, ovarian cancer, uterine cancer, prostate cancer, testicular germ cell tumor, brain cancer, gastric cancer, esophagus cancer, lung cancer, liver cancer, renal cell cancer and colon cancer.

49. The method of any of claim 45-48, wherein the effective amount is effective to reduce tumor size.

50. A method of making hydroxyl dendrimers covalently conjugated via ether linkages to one or more positron emission tomography (PET) imaging agents or one or more magnetic resonance imaging (MRI) contrast agents, the method comprising etherification of one or more surface groups of the dendrimers prior to conjugation the one or more surface groups of the dendrimers to one or more PET imaging agents, optionally via one or more spacers, and wherein the conjugation is via an ether linkage.

51. The method of claim 50, wherein the PET imaging agent or the MRI contrast agent comprises a chelator selected from the group consisting of p-SCN-Bn-Deferoxamine, diethylene triamine pent-acetic acid (DTPA), 1,4,7,10-tetraazacyclododecane-1,4,7,10-tetraacetic acid (DOTA), diamine dithiols, activated mercaptoacetyl-glycyl-glycyl-glycine (MAG3), and hydrazidonicotinamide (HYNIC).

52. The method of claim 50 or claim 51, wherein the PET imaging agent comprises radionuclides selected from the group consisting of ⁶⁴Cu, ⁸⁹Zr, ⁹⁰Y, ¹⁰⁵R, ¹¹¹In, ^{117m}Sn, ¹⁴⁹Pm, ¹⁵³Sm, ¹⁶¹Tb, ¹⁶⁶Tb, ¹⁶⁶Dy, ¹⁶⁶Ho, ¹⁷⁵Yb, ¹⁷⁷Lu, ²²⁵Ac, ^{186/188}Re, and ¹⁹⁹Au.

53. The method of claim 50 or claim 51, wherein the MRI contrast agent comprises metals selected from the group consisting of Gd, Mn, BaSO₄, iron oxides, and iron platinum.

54. The method of any one of claims 50-53, wherein the dendrimer is further complexed and/or conjugated to one or more therapeutic, prophylactic, and/or diagnostic agents.

55. A method for treating a neurodegenerative disorder comprising administering to a subject in need thereof an effective amount of a composition comprising a compound that comprises a dendrimer conjugated to a radionuclide or an MRI contrast agent through an ester, ether, or amide linkage, wherein the dendrimer comprises a high density of surface hydroxyl groups.

56. The method of claim 55, wherein the neurodegenerative disorder is Alzheimer's disease.

57. The method of claim 55, wherein the neurodegenerative disorder is ALS.
